SEQUENCE LISTING (1 sequence)

SEQ ID NO: 1 - protein, 12 aa, Artificial Sequence
  Description of Artificial Sequence Synthetic peptide
XSWSFXRRRQRR

(19) United States
(12) Patent Application Publication
(10) Pub. No.: US 2010/0112089 A1
(43) Pub. Date: May 6, 2010

(54) PEPTIDE COMPOUND AND USE THEREOF

(75) Inventors: Takumi Kawabe, Shizuoka (JP); Machiyo Ishigaki, Shizuoka (JP); Takuji Sato, Shizuoka (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Takeda Pharmaceutical Company Limited

(21) Appl. No.: 12/465,536

(22) Filed: May 13, 2009

Related U.S. Application Data
(60) Provisional application No. 61/053,190, filed on May 14, 2008.
(60) Provisional application No. 61/116,849, filed on Nov. 21, 2008.

(51) Int. Cl. A61K 38/10; C07K 7/00; A61K 38/14; A61K 33/24; A61P 35/00

(52) U.S. Cl. 424/649; 530/327; 514/14; 514/8

(57) ABSTRACT

The present invention provides a method for the prophylaxis or treatment of cancer in a mammal, comprising administering a therapeutically effective amount of CBP501, a prodrug thereof or a pharmaceutically acceptable salt thereof to the mammal, wherein CBP501, a prodrug thereof or a pharmaceutically acceptable salt thereof is administered simultaneously with or before administration of a nucleic acid damaging agent.

This application claims benefit of U.S. Provisional Patent Application No. 61/053,190, filed May 14, 2008, and U.S. Provisional Patent Application No. 61/116,849, filed Nov. 21, 2008, the contents of each of which are incorporated herein by reference.

### TECHNICAL FIELD

The present invention relates to a peptide compound and the use thereof. More specifically, the present invention relates to a peptide compound having an anti-tumor activity and the use thereof, and combination therapies using the compound.

### BACKGROUND OF THE INVENTION

In cancer cells, loss of the G1 checkpoint renders the cells more dependent on the G2 checkpoint to repair damaged DNA. Since normal cells mostly repair DNA at the G1 checkpoint, it is anticipated that disruption of the G2 checkpoint would have a greater impact on tumor cells with an impaired G1 checkpoint than on normal cells with an intact G1 checkpoint (non-patent literatures 10, 19 and 20).

CBP501 is a novel cell cycle G2 checkpoint abrogator. It is a stable synthetic dodecapeptide which inhibits the activities of several kinases involved in G2 arrest (FIG. 1). CBP501 inhibits the phosphorylation of Cdc25C, thus inhibiting the binding of 14-3-3 to this site. The phosphorylation of Cdc25C and/or the binding of 14-3-3 prevents Cdc25C from activating CDC2/Cyclin B, a master switch for the G2 to M phase transition (non-patent literatures 21-23).

DNA damaging agents, such as cisplatin, bleomycin, and camptothesin, have been widely used to treat cancer patients, and treatment with these agents triggers the cellular response to DNA damage, including cell cycle arrest, DNA repair, and apoptosis. The DNA damage response pathways are induced by direct or indirect recognition of damaged DNA by sensor proteins, such as MRN, MRE11-RAD50-NBS1 (non-patent literature 1) and 9-1-1, RAD9-RAD1-HUS1 (non-patent literature 2) sensor protein complexes as well as 53BP1 (non-patent literature 3), MDC1 (non-patent literature 4), SMC1 (non-patent literature 5) and TopBP1 (non-patent literature 6). Recruitment of the sensor proteins to the DNA double strand break depends on the phosphorylation of histone H2AX, namely γ-H2AX (non-patent literature 7). These sensor proteins can serve as markers for DNA damage, and can be visualized via confocal immunofluorescence microscopy as damage-induced foci. The sensor proteins then induce pathways that activate ATM and ATR, which activate CHK1 and CHK2, resulting in cell cycle arrest (non-patent literature 8). MAPKAP-K2 can also be activated by DNA damage in specific conditions, including in response to damage caused by cisplatin and UV irradiation (non-patent literature 9). These downstream kinases, CHK1, CHK2 or MAPKAP-K2, phosphorylate several residues in CDC25s, including serine 216 of CDC25C, and cause a G2 phase cell cycle arrest in many cancer cells, as the majority of cancer cells do not have an intact G1 checkpoint (non-patent literature 10). Phosphorylation of CDC25C prevents it from activating CDC2/Cyclin B, a master regulator of the transition from G2 to M phase (non-patent literature 11).

CBP501 is a synthetic peptide that was identified by a cell cycle phenotype-based optimization of TAT-S216A (non-patent literature 12). TAT-S216A is a G2 checkpoint abrogating fusion peptide that contains a sequence surrounding serine 216 of CDC25C, functioning as a substrate mimic inhibitor of kinases that phosphorylate serine 216, and an HIV-TAT sequence (non-patent literature 13), functioning as a carrier for trans-membrane transduction. The optimization identified a peptide that reduced the G2 phase population in bleomycin treated Jurkat cells without any effect on cell cycle progression or cell cycle distribution of normal cells and of the cells treated with colchicine. CBP501 was found to inhibit multiple kinases that phosphorylate serine 216 of CDC25C, such as MAPKAP-K2, CHK1, C-TAK1 and, to a lesser extent, CHK2, and subsequently reduce phosphorylation of serine 216 on CDC25C (non-patent literature 14), consistent with its observed function as a cell cycle G2 checkpoint abrogator. CBP501 is now in clinical study (non-patent literature 15).

### CITATION LIST

non-patent literature 1: Lavin M F. ATM and the Mrell complex is combine to recognize and signal DNA double-strand breaks. Oncogene 2007; 26:7749-58.
non-patent literature 2: Helt C E, Wang W, Keng P C, Bambara R A. Evidence that DNA damage detection machinery participates in DNA repair. Cell Cycle 2005; 4:529-32.
non-patent literature 3: Adams M M, Carpenter P B. Tying the loose ends together in DNA double strand break repair with 53BP1. Cell Div 2006; 1:19.
non-patent literature 4: Kim J E, Minter-Dykhouse K, Chen J. Signaling networks controlled by the MRN complex and MDC1 during early DNA damage responses. Mol Carcinog 2006; 45:403-8.
non-patent literature 5: Kitagawa R, Kastan M B. The ATM-dependent DNA damage signaling pathway. Cold Spring Harb Symp Quant Biol 2005; 70:99-109.
non-patent literature 6: Garcia V, Furuya K, Carr A M. Identification and functional analysis of TopBP1 and its homologs. DNA Repair 2005; 214:1227-39.
non-patent literature 7: Kuo L J, Yang L X. Gamma-H2AX—a novel biomarker for DNA double-strand breaks. In Vivo 2008; 22:305-9.
non-patent literature 8: Sancar A, Lindsey-Boltz L A, Unsal-Kaçmaz K, Linn S. Molecular mechanisms of mammalian DNA repair and the DNA damage checkpoints. Annu Rev Biochem 2004; 73:39-85.
non-patent literature 9: Reinhardt H C, Aslanian A S, Lees J A, Yaffe M B. p53-deficient cells rely on ATM- and ATR-mediated checkpoint signaling through the p38MAPK/MK2 pathway for survival after DNA damage. Cancer Cell 2007; 2:175-89.
non-patent literature 10: Kawabe T. G2 checkpoint abrogators as anticancer drugs. Mol Cancer Ther 2004; 3:513-9.
non-patent literature 11: Hutchins J R, Clarke P R. Many fingers on the mitotic trigger: post-translational regulation of the Cdc25C phosphatase. Cell Cycle 2004; 3:41-5.
non-patent literature 12: Suganuma M, Kawabe T, Hori H, Funabiki T, Okamoto T. Sensitization of cancer cells to DNA damage-induced cell death by specific cells cycle G2 checkpoint abrogation. Cancer Res 1999; 59:5887-91.
non-patent literature 13: Nagahara H, Vocero-Akbani A M, Snyder E L, et al. Transduction of full-length TAT fusion proteins into mammalian cells: TAT-p27Kip1 induces cell migration. Nat Med 1998; 12:1449-52.
non-patent literature 14: Sha S K, Sato T, Kobayashi H, et al. Cell cycle phenotype-based optimization of G2-abrogating peptides yields CBP501 with a unique mechanism of action at the G2 checkpoint. Mol Cancer Ther 2007; 6:147-53.
non-patent literature 15: Wong B Y, Shapiro G, Gordon M S, et al. Phase I studies of CBP501, a novel G2 checkpoint abrogator, alone and combined with cisplatin (CDDP) in advanced solid tumor patients (pts). 2008; ASCO Abstract #2528.
non-patent literature 16: Zhang P, Gao W, Li H, Reed E, Chen F. Inducible degradation of checkpoint kinase 2 links to cisplatin-induced resistance in ovarian cancer cells. Biochem Biophys Res Commun 2005; 328:567-72.
non-patent literature 17: Kass E M, Ahn J, Tanaka T, Freed-Pastor W A, Keezer S, Prives C. Stability of checkpoint kinase 2 is regulated via phosphorylation at serine 456. J Biol Chem 2007; 282:30311-21.
non-patent literature 18: Lovly C M, Yan L, Ryan C E, Takada S, Piwnica-Worms H. Regulation of Chk2 ubiquitination and signaling through autophosphorylation of serine 379. Mol Cell Biol 2008; 28:5874-85.
non-patent literature 19: Hartwell L, Kasten M. Cell cycle control and cancer. Science 1994; 266:1821-1828.
non-patent literature 20: Levine A J. p 53, the cellular gatekeeper for growth and division. Cell 1997; 88:323-331.
non-patent literature 21: Peng C Y, Graves P R, Thoma R S, et al. Mitotic and G2 checkpoint control: regulation of 14-3-3 protein binding by phosphorylation of Cdc25C on serine-216. Science 1997; 277:1501-1505.
non-patent literature 22: Lopez-Girona A, Furnari B, Mondesert O, et al. Nuclear localization of Cdc25 is regulated by DNA damage and a 14-3-3 protein. Nature 1999; 397:172-175.
non-patent literature 23: Blasina A, de Weyer I, Laus M C, et al. A human homologue of the checkpoint kinase Cds1 directly inhibits Cdc25 phosphatase. Curr Biol 1999; 9:1-10.

### SUMMARY OF THE INVENTION

The present inventors have conducted intensive studies of G2 checkpoint abrogator and found that a combined use of a peptide compound (CBP501) having a structure shown by the following sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg)(SEQ ID NO:1) and a DNA damaging agent affords a more effective anti-cancer treatment causing less side effects.

Here the present inventors show an additional activity of CBP501 in enhancing the cytotoxicity of bleomycin and/or platinum-containing drug (e.g., cisplatin) by increasing DNA damage, confirmed by enhanced formation of foci containing ATM, NBS1, DNA-PKcs, SMC1, and γ-H2AX, as well as increased checkpoint signals.

Accordingly, the present invention provides the following.

[1] An acetate salt of a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1).
[2] An agent for the prophylaxis or treatment of a cell proliferative disorder, comprising the acetate salt of the peptide compound of the above-mentioned [1] as an active ingredient.
[3] The agent of the above-mentioned [2], wherein the cell proliferative disorder is at least one selected from the group consisting of breast cancer, prostate cancer, pancreas cancer, gastric cancer, lung cancer, pleural mesothelioma, colon cancer, rectal cancer, large bowel cancer, small intestinal cancer, esophageal cancer, duodenal cancer, lingual cancer, pharyngeal cancer, salivary gland cancer, cerebral tumor, schwanoma, liver cancer, kidney cancer, bile duct cancer, endometrial cancer, cervical cancer, uterine body cancer, ovarian cancer, bladder cancer, urethral cancer, skin cancer, angioma, malignant lymphoma, malignant melanoma, thyroid cancer, parathyroid cancer, nasal cancer, paranasal cancer, auditory organ cancer, carcinoma of oral floor, laryngeal cancer, unknown primary cancer, parotid cancer, submandibular cancer, bone tumor, angiofibroma, retinal sarcoma, penile cancer, testicular tumor, pediatric solid cancer, Kaposi's sarcoma, Kaposi's sarcoma resulted from AIDS, tumor of maxillary sinus, fibrous histiocytoma, leiomyosarcoma, rhabdomyosarcoma, multiple myeloma and leukemia.
[4] The agent of the above-mentioned [2], wherein the cell proliferative disorder is at least one selected from the group consisting of endometrial cancer, peritoneal mesothelioma, pericardial mesothelioma, uterine body cancer and ovarian cancer.
[5] A method of producing an acetate salt of a peptide compound shown by SEQ ID No:1, comprising a step of performing liquid chromatography using an acetate-containing solvent.
[6] An agent for the prophylaxis or treatment of at least one disease selected from the group consisting of endometrial cancer, peritoneal mesothelioma and pericardial mesothelioma, comprising a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof, or a pharmaceutically acceptable salt thereof as an active ingredient.
[7] A pharmaceutical composition comprising an acetate salt of a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1) and a nucleic acid damaging agent.
[8] The pharmaceutical composition of the above-mentioned [7], which is used for the prophylaxis or treatment of cell proliferative disorders.
[9] The pharmaceutical composition of the above-mentioned [8], wherein the cell proliferative disorder is at least one selected from the group consisting of breast cancer, prostate cancer, pancreas cancer, gastric cancer, lung cancer, pleural mesothelioma, colon cancer, rectal cancer, large bowel cancer, small intestinal cancer, esophageal cancer, duodenal cancer, lingual cancer, pharyngeal cancer, salivary gland cancer, cerebral tumor, schwanoma, liver cancer, kidney cancer, bile duct cancer, endometrial cancer, cervical cancer, uterine body cancer, ovarian cancer, bladder cancer, urethral cancer, skin cancer, angioma, malignant lymphoma, malignant melanoma, thyroid cancer, parathyroid cancer, nasal cancer, paranasal cancer, auditory organ cancer, carcinoma of oral floor, laryngeal cancer, unknown primary cancer, parotid cancer, submandibular cancer, bone tumor, angiofibroma, retinal sarcoma, penile cancer, testicular tumor, pediatric solid cancer, Kaposi's sarcoma, Kaposi's sarcoma resulted from AIDS, tumor of maxillary sinus, fibrous histiocytoma, leiomyosarcoma, rhabdomyosarcoma, multiple myeloma and leukemia.
[10] The pharmaceutical composition of the above-mentioned [8], wherein the cell proliferative disorder is at least one selected from the group consisting of endometrial cancer, peritoneal mesothelioma, pericardial mesothelioma, uterine body cancer and ovarian cancer.
[11] The pharmaceutical composition of any of the above-mentioned [7] to [10], wherein the nucleic acid damaging agent is at least one selected from the group consisting of bleomycins and a platinum-containing drug.
[12] The pharmaceutical composition of any of the above-mentioned [7] to [10], wherein the nucleic acid damaging agent is at least one selected from the group consisting of bleomycin, cisplatin, carboplatin and oxaliplatin.
[13] The pharmaceutical composition of any of the above-mentioned [7] to [10], wherein the nucleic acid damaging agent is cisplatin.
[14] The pharmaceutical composition of any of the above-mentioned [10] to [13], further comprising pemetrexed.
[15] The pharmaceutical composition of the above-mentioned [14], which is used for the treatment of peritoneal mesothelioma.
[16] The pharmaceutical composition of any of the above-mentioned [10] to [13], further comprising gemcitabine.
[17] The pharmaceutical composition of the above-mentioned [16], which is used for the treatment of pancreas cancer.
[18] An agent for the prophylaxis or treatment of a cell proliferation disorder, comprising a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof as an active ingredient, which is administered simultaneously with or before a nucleic acid damaging agent.
[19] The agent of the above-mentioned [18], wherein the pharmaceutically acceptable salt is an acetate salt.
[20] The agent of the above-mentioned [18] or [19], wherein the cell proliferative disorder is at least one selected from the group consisting of breast cancer, prostate cancer, pancreas cancer, gastric cancer, lung cancer, pleural mesothelioma, colon cancer, rectal cancer, large bowel cancer, small intestinal cancer, esophageal cancer, duodenal cancer, lingual cancer, pharyngeal cancer, salivary gland cancer, cerebral tumor, schwanoma, liver cancer, kidney cancer, bile duct cancer, endometrial cancer, cervical cancer, uterine body cancer, ovarian cancer, bladder cancer, urethral cancer, skin cancer, angioma, malignant lymphoma, malignant melanoma, thyroid cancer, parathyroid cancer, nasal cancer, paranasal cancer, auditory organ cancer, carcinoma of oral floor, laryngeal cancer, unknown primary cancer, parotid cancer, submandibular cancer, bone tumor, angiofibroma, retinal sarcoma, penile cancer, testicular tumor, pediatric solid cancer, Kaposi's sarcoma, Kaposi's sarcoma resulted from AIDS, tumor of maxillary sinus, fibrous histiocytoma, leiomyosarcoma, rhabdomyosarcoma, multiple myeloma and leukemia.
[21] The agent of the above-mentioned [18] or [19], wherein the cell proliferative disorder is at least one selected from the group consisting of endometrial cancer, peritoneal mesothelioma, pericardial mesothelioma, uterine body cancer and ovarian cancer.
[22] The agent of any of the above-mentioned [18] to [21], wherein the nucleic acid damaging agent is at least one selected from the group consisting of bleomycins and a platinum-containing drug.
[23] The agent of any of the above-mentioned [18] to [21], wherein the nucleic acid damaging agent is at least one selected from the group consisting of bleomycin, cisplatin, carboplatin and oxaliplatin.
[24] The agent of any of the above-mentioned [18] to [21], wherein the nucleic acid damaging agent is cisplatin.
[25] The agent of any of the above-mentioned [22] to [24], which is used in combination with pemetrexed.
[26] The agent of the above-mentioned [25], which is used for the treatment of peritoneal mesothelioma.
[27] The agent of any of the above-mentioned [22] to [24], which is used in combination with gemcitabine.
[28] The agent of the above-mentioned [27], which is used for the treatment of pancreas cancer.
[29] An agent for the prophylaxis or treatment of a cell is proliferation disorder, comprising a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof as an active ingredient, which is administered after administration of a nucleic acid damaging agent.
[30] The agent of the above-mentioned [29], wherein the pharmaceutically acceptable salt is an acetate salt.
[31] The agent of the above-mentioned [29] or [30], wherein the cell proliferative disorder is at least one selected from the group consisting of breast cancer, prostate cancer, pancreas cancer, gastric cancer, lung cancer, pleural mesothelioma, colon cancer, rectal cancer, large bowel cancer, small intestinal cancer, esophageal cancer, duodenal cancer, lingual cancer, pharyngeal cancer, salivary gland cancer, cerebral tumor, schwanoma, liver cancer, kidney cancer, bile duct cancer, endometrial cancer, cervical cancer, uterine body cancer, ovarian cancer, bladder cancer, urethral cancer, skin cancer, angioma, malignant lymphoma, malignant melanoma, thyroid cancer, parathyroid cancer, nasal cancer, paranasal cancer, auditory organ cancer, carcinoma of oral floor, laryngeal cancer, unknown primary cancer, parotid cancer, submandibular cancer, bone tumor, angiofibroma, retinal sarcoma, penile cancer, testicular tumor, pediatric solid cancer, Kaposi's sarcoma, Kaposi's sarcoma resulted from AIDS, tumor of maxillary sinus, fibrous histiocytoma, leiomyosarcoma, rhabdomyosarcoma, multiple myeloma and leukemia.
[32] The agent of the above-mentioned [29] or [30], wherein the cell proliferative disorder is at least one selected from the group consisting of endometrial cancer, peritoneal mesothelioma, pericardial mesothelioma, uterine body cancer and ovarian cancer.
[33] The agent of any of the above-mentioned [29] to [32], wherein the nucleic acid damaging agent is carboplatin or oxaliplatin.
[34] A method for the prophylaxis or treatment of a cell proliferation disorder in a mammal, comprising administering a therapeutically effective amount of a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof to the mammal simultaneously with or before a nucleic acid damaging agent.
[35] A method for the prophylaxis or treatment of a cell proliferation disorder in a mammal, comprising administering a therapeutically effective amount of a peptide compound shown by the sequence (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof to the mammal before a nucleic acid damaging agent.
[36] The method of the above-mentioned [34] or [35], wherein the pharmaceutically acceptable salt is acetate salt.
[37] The method of any of the above-mentioned [34] to [36], wherein the cell proliferative disorder is at least one selected from the group consisting of breast cancer, prostate cancer, pancreas cancer, gastric cancer, lung cancer, pleural mesothelioma, colon cancer, rectal cancer, large bowel cancer, small intestinal cancer, esophageal cancer, duodenal cancer, lingual cancer, pharyngeal cancer, salivary gland cancer, cerebral tumor, schwanoma, liver cancer, kidney cancer, bile duct cancer, endometrial cancer, cervical cancer, uterine body cancer, ovarian cancer, bladder cancer, urethral cancer, skin cancer, angioma, malignant lymphoma, malignant melanoma, thyroid cancer, parathyroid cancer, nasal cancer, paranasal cancer, auditory organ cancer, carcinoma of oral floor, laryngeal cancer, unknown primary cancer, parotid cancer, submandibular cancer, bone tumor, angiofibroma, retinal sarcoma, penile cancer, testicular tumor, pediatric solid is cancer, Kaposi's sarcoma, Kaposi's sarcoma resulted from AIDS, tumor of maxillary sinus, fibrous histiocytoma, leiomyosarcoma, rhabdomyosarcoma, multiple myeloma and leukemia.
[38] The method of any of the above-mentioned [34] to [36], wherein the cell proliferative disorder is at least one selected from the group consisting of endometrial cancer, peritoneal mesothelioma, pericardial mesothelioma, uterine body cancer and ovarian cancer.
[39] The method of any of the above-mentioned [34] to [38], wherein the nucleic acid damaging agent is at least one selected from the group consisting of bleomycins and a platinum-containing drug.
[40] The method of any of the above-mentioned [34] to [38], wherein the nucleic acid damaging agent is at least one selected from the group consisting of bleomycin, cisplatin, carboplatin and oxaliplatin.
[41] The method of any of the above-mentioned [34] to [38], wherein the nucleic acid damaging agent is cisplatin.
[42] The method of any of the above-mentioned [39] to [41], further comprising an administration of pemetrexed.
[43] The method of the above-mentioned [42], wherein the cell proliferative disorder is peritoneal mesothelioma.
[44] The method of any of the above-mentioned [39] to [41], further comprising an administration of gemcitabine.
[45] The method of the above-mentioned [44], wherein the cell proliferative disorder is pancreas cancer.
[46] A method for the prophylaxis or treatment of a cell proliferation disorder in a mammal, comprising performing the following step a) and step b) as one cycle once a week for 3 weeks;
a) step of administering a therapeutically effective amount of a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof to a mammal by intravenous infusion, and
b) step of administering a therapeutically effective amount of cisplatin to the mammal after completion of step a).
[47] A method for the prophylaxis or treatment of a cell proliferation disorder in a mammal, comprising performing the following step a) and step b) as one cycle once a day for 5 consecutive days;
a) step of administering a therapeutically effective amount of a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof to a mammal by intravenous infusion, and
b) step of administering a therapeutically effective amount of cisplatin to the mammal after completion of step a).
[48] A method for the prophylaxis or treatment of a cell proliferation disorder in a mammal, comprising performing the following step a)-step c) as one cycle every 3 weeks;
a) step of administering a therapeutically effective amount of a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof to a mammal by intravenous infusion,
b) step of administering a therapeutically effective amount of pemetrexed to the mammal after completion of step a), and
c) step of administering a therapeutically effective amount of cisplatin to the mammal after completion of step b).
[49] A method for the prophylaxis or treatment of a cell proliferation disorder in a mammal, comprising administering a therapeutically effective amount of a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof to the mammal after administration of a nucleic acid damaging agent.
[50] The method of the above-mentioned [49], wherein the pharmaceutically acceptable salt is an acetate salt.
[51] The method of the above-mentioned [49] or [50], wherein the cell proliferative disorder is at least one selected from the group consisting of breast cancer, prostate cancer, pancreas cancer, gastric cancer, lung cancer, pleural mesothelioma, colon cancer, rectal cancer, large bowel cancer, small intestinal cancer, esophageal cancer, duodenal cancer, lingual cancer, pharyngeal cancer, salivary gland cancer, cerebral tumor, schwanoma, liver cancer, kidney cancer, bile duct cancer, endometrial cancer, cervical cancer, uterine body cancer, ovarian cancer, bladder cancer, urethral cancer, skin cancer, angioma, malignant lymphoma, malignant melanoma, thyroid cancer, parathyroid cancer, nasal cancer, paranasal cancer, auditory organ cancer, carcinoma of oral floor, laryngeal cancer, unknown primary cancer, parotid cancer, submandibular cancer, bone tumor, angiofibroma, retinal sarcoma, penile cancer, testicular tumor, pediatric solid cancer, Kaposi's sarcoma, Kaposi's sarcoma resulted from AIDS, tumor of maxillary sinus, fibrous histiocytoma, leiomyosarcoma, rhabdomyosarcoma, multiple myeloma and leukemia.
[52] The method of the above-mentioned [49] or [50], wherein the cell proliferative disorder is at least one selected from the group consisting of endometrial cancer, peritoneal mesothelioma, pericardial mesothelioma, uterine body cancer and ovarian cancer.
[53] The method of any of the above-mentioned [49] to [52], wherein the nucleic acid damaging agent is carboplatin or oxaliplatin.
[54] An agent for potentiating a cell proliferation is suppressive action of a platinum-containing preparation, comprising a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof as an active ingredient.
[55] The agent of the above-mentioned [54], wherein the platinum-containing drug is cisplatin, carboplatin or oxaliplatin.
[56] The agent of the above-mentioned [54], wherein the platinum-containing drug is cisplatin.
[57] The agent of any of the above-mentioned [54] to [56], comprising using pemetrexed in combination.
[58] The agent of any of the above-mentioned [54] to [56], comprising using gemcitabine in combination.
[59] A method for potentiating a cell proliferation suppressive action of a platinum-containing preparation, comprising administering, to a mammal, a therapeutically effective amount of a peptide compound shown by the sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof as an active ingredient.
[60] The method of the above-mentioned [59], wherein the platinum-containing drug is cisplatin, carboplatin or oxaliplatin.
[61] The method of the above-mentioned [59], wherein the platinum-containing drug is cisplatin.
[62] The method of any of the above-mentioned [59] to [61], comprising using pemetrexed in combination.
[63] The method of any of the above-mentioned [59] to [61], comprising using gemcitabine in combination.

### BRIEF DESCRIPTION OF THE DRAWINGS

FIG. 1 shows the scheme of cell cycle G2 checkpoint-related signal cascade.

FIG. 2 shows the scheme of the dosing schedule of the multi-center dose escalation studies with different treatment regimens.

FIG. 3 shows evolution of the tumor marker CA-125 in two patients. In a patient with endometrial carcinoma, treated in the combination study at 24.3 mg/m² CBP501/75 mg/m² cisplatin, CA-125 was reduced from 1117 U/mL at baseline to a nadir of 350 U/mL at cycle 9; this patient also experienced a confirmed partial response according to RECIST criteria. In a patient with ovarian adenocarcinoma (36.4 mg/m² CBP501/75 mg/m² cisplatin), CA-125 was 75 U/mL at baseline, and reached a nadir of 31 U/mL at cycle 4; levels of the tumor marker were found to increase from cycle 5 onwards. This patient experienced a best tumor response of SD (stable disease) according to RECIST criteria. RECIST: response evaluation criteria in solid tumours

FIG. 4 shows characteristic CBP501 concentration-time profile.

Small diamond=Day 1 of treatment
Rectangular=Day 15 of treatment

FIG. 5 shows mean CBP501 AUC_{O-inf}. Bars are standard deviation, lines are linear interpolation.

FIG. 6 shows mean CBP501 Cₘₐₓ. Bars are standard deviation, lines are linear interpolation.

FIG. 7 shows the dose dependent increase of the cells in G2/M phase in response to treatment with cisplatin in the presence or absence of CBP501. Y-axis: % of cells at 4N DNA content as determined by FACS analysis, X-axis: doses of cisplatin in μg/ml. FIG. 7a. Cells were treated with the indicated doses of CBP501 for 3 hr on day 1, and the indicated dose of cisplatin for 3 hr on day 2, and then analyzed by FACS on day 4. FIG. 7b. Cells were treated with the indicated doses of cisplatin and CBP501 for 3 hr on day 1 and analyzed by FACS on day 3.

FIG. 8 shows the quantitative analysis of platinum in the cell and on DNA. FIG. 8a. Platinum concentration in cells treated with cisplatin and CBP501. NCI-H226 cells were treated with cisplatin in the presence or absence of CBP501 at the indicated doses for 3 hr and harvested immediately after treatment (left panel) or after an additional 48 hr of culture without cisplatin and CBP501 (right panel). X-axis: amount of cisplatin, Y-axis: platinum concentration per cell in ng/10⁵ cells. FIG. 8b. Platinum amount on the extracted genomic DNA in MIAPaCa2, HT-29 and HUVEC cells. MIAPaCa2, HT29, and HUVEC cells were treated with 10 μM of CBP501 and 3 μg/ml of cisplatin for 3 hr. The quantity of platinum of the genomic DNA was determined by ICP-MS.

FIG. 9 shows foci formation determined by confocal immuno-fluorescence microscopy in cells treated with bleomycin in the presence or absence of CBP501 for 3 hr. FIG. 9a. NCI-H226 cells treated with (BLM) or without (NT) bleomycin in the absence or presence of CBP501. Green fluorescence (high brightness region) indicates the specific antibody staining and the nuclei were counter stained with Hoechst 33342 (blue fluorescence; low brightness region). FIG. 9b. Number of foci in NCI-H226 cells treated with bleomycin in the absence or presence of CBP501.

FIG. 10 shows western blot analysis of the DNA damage sensor and G2 checkpoint related proteins in NCI-H226 cells treated with DNA damaging agents and CBP501. NCI-H226 cells were treated with 2 μg/ml bleomycin (FIG. 10a) or 10 μg/ml cisplatin (FIG. 10b) with or without 2 μM of CBP501 and harvested at the indicated time points. The membrane was probed with the indicated antibodies.

FIG. 11 shows WST analysis of NCI-H226 and MSTO-211H cells. Cells were treated with the indicated amounts of is cisplatin and CBP501 and cultured for 72 hours. Experiments were performed in triplicate. Error bar: standard deviation of the results.

FIG. 12 shows in vivo activity of cisplatin and CBP501 in the subcutaneous xenograft tumor model of NCI-H226. CBP501 (7.5 mg/kg) was intravenously bolus injected twice on day 1 and once on day 2 to the indicated groups. Cisplatin was injected intraperitoneally once on day 2 to the indicated groups.

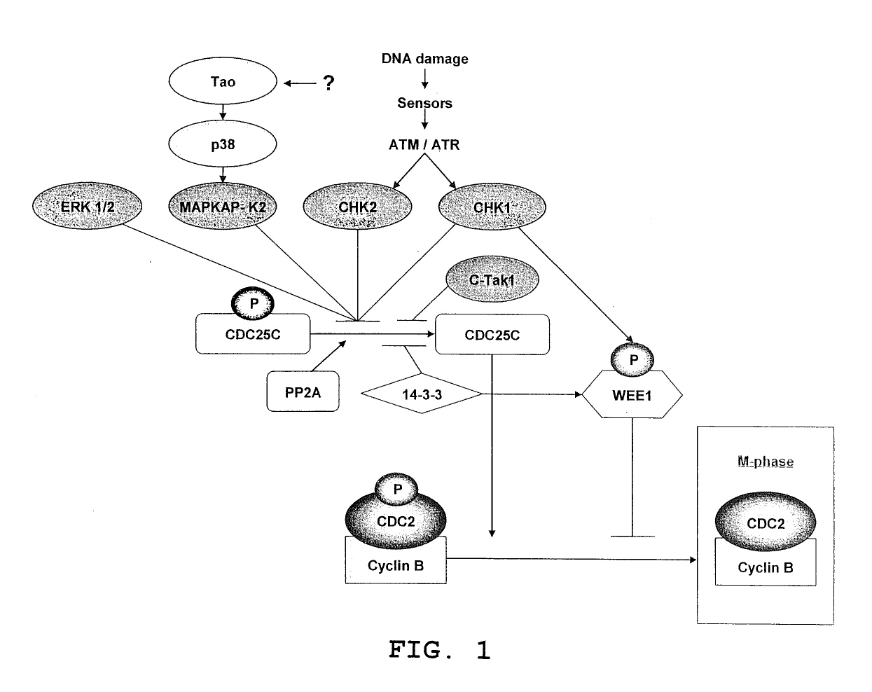

### DESCRIPTION OF EMBODIMENTS

In the present invention, a peptide compound having the following structure: (d-Bpa)(d-Ser)(d-Trp)(d-Ser) (d-Phe-2,3,4,5,6-F)(d-Cha)(d-Arg)(d-Arg)(d-Arg)(d-Gln)(d-Arg)(d-Arg) (SEQ ID NO:1; CBP501) is used.

CBP501 inhibits cell proliferation. CBP501 is therefore useful for treating cell proliferative disorders or physiological conditions characterized by undesirable or unwanted cell proliferation, such as benign and malignant tumor cells. The ability of CBP501 to inhibit cell proliferation appears to be due at least in part to abrogation of the cell cycle G2 checkpoint. Because cells can be induced to enter the cell cycle G2 checkpoint in response to nucleic acid damage to allow the cell to repair the damage before DNA replication and cell division occurs, by inhibiting the G2 checkpoint, CBP501 sensitize cells to nucleic acid damaging agents and treatment protocols. Cells that accumulate enough nucleic acid damage will be unable to complete repair of the damaged nucleic acid because the G2 checkpoint is disrupted. Such cells will exhibit decreased proliferation (e.g., due to mutation of a gene critical for survival that is not repaired) and eventually undergo apoptosis.

Cells having a normal G1 are less susceptible to accumulating damaged nucleic acid since nucleic acid repair can also take place during G1. Thus, normal cells are less susceptible to the effects of CBP501. However, cells having an impaired or disrupted cell cycle G1 checkpoint are more likely to accumulate damaged nucleic acid because the G1 checkpoint is impaired or disrupted making it less likely that the cells can completely repair the damaged nucleic acid. Thus, treating G1 impaired or disrupted cells with CBP501 that disrupts the G2 checkpoint makes the cells even less likely to be able to complete repair of the damaged nucleic acid. G1 impaired or disrupted cells are therefore particularly sensitive to CBP501. Thus, CBP501 can be used to inhibit or prevent cell proliferation in general and in particular inhibit proliferation of cells having an impaired or disrupted G1 checkpoint.

Cells having an impaired or disrupted G1 cell cycle checkpoint include but are not limited to cells that rapidly proliferate. Cell proliferative disorders and physiological conditions characterized by rapidly growing cells, undesirably growing cells or cells that survive instead of undergoing apoptosis frequently have impaired or disrupted G1 cell cycle checkpoint. Thus, as it appears that the ability of CBP501 to inhibit proliferation or stimulate apoptosis is due, at least in part, to disrupting the G2 cell cycle checkpoint, cells that rapidly or undesirably proliferate due to an impaired or disrupted G1 checkpoint are particularly attractive targets.

CBP501 may also suppress cell proliferation by themselves without additional treatments that damage nucleic acid or that have anti-proliferative activity since disrupting G2 checkpoint will likely lead to the accumulation of nucleic acid damage as the cells divide. Accordingly, abnormal or undesirably proliferating or surviving cells can be treated with CBP501 alone, or in combination with a nucleic acid damaging treatment (e.g., a chemical agent or treatment protocol), to inhibit or prevent proliferation of the cells or to stimulate cell apoptosis/catastrophe.

Preferably, the treatment with CBP501 is performed simultaneously with or before the nucleic acid damaging treatment. More preferably, the treatment with CBP501 is performed before a nucleic acid damaging treatment.

Unlike conventional anti-cell proliferative agents, which target rapidly proliferating cells irrespective of whether the cells are normal or abnormal (e.g., cancer cell), CBP501 preferentially target cells having an impaired or disrupted cell cycle G1 checkpoint. CBP501 is less likely to produce excess undesirable side effects associated with conventional anti-cell proliferative treatment, such as bone marrow suppression, nausea, loss of appetite, diarrhea, and hair loss. In addition, because the vast majority of cancer cells have an impaired or disrupted cell cycle G1 checkpoint, cancer cells will exhibit increased sensitivity to CBP501 that abrogate cell cycle G2 checkpoint. That normal cells are less susceptible also means that CBP501 can be used in greater amounts.

In the present invention, CBP501 having anti-cell proliferative activity and/or that abrogate the G2 cell cycle checkpoint are used. CBP501 include sequences that inhibit proliferation of a cell or that stimulate apoptosis of a cell. CBP501 also include sequences that abrogate cell cycle G2 checkpoint.

In the present invention, a novel activity of CBP501 is increasing the cytotoxicity caused by nucleic acid damaging treatment (e.g., a chemical agent (bleomycins such as bleomycin, platinum-containing drug such as cisplatin etc.) or treatment protocol). CBP501 enhanced damage-induced formation of foci and DNA damage checkpoint signals in bleomycin treated cells. CBP501 increased the number of cells in G2, as well as the platinum concentration, platinum-DNA adducts and checkpoint signals in cisplatin treated cells. This phenomenon was observed in a variety of cancer cell lines, including all four tested malignant pleural mesothelioma cell lines and MIAPaCa2, a pancreatic cancer cell line, but not human umbilical vein endothelial (HUVEC) cells.

CBP501 enhanced the in vitro cytotoxicity and in vivo anti-tumor activity of nucleic acid damaging treatment.

Like CBP501, various derivatives and analogs thereof are also used preferably. Examples of the derivatives are those recited in U.S. Pat. No. 6,995,135. As mentioned below, like CBP501, a prodrug thereof and pharmaceutically acceptable salts thereof are also used preferably. Unless otherwise specified, a simple reference to CBP501 embraces the entirety of such series of compounds.

As discussed, CBP501 have anti-cell proliferative activity or G2 abrogating activity alone. Anti-cell proliferative activity can be increased by combining CBP501 with treatments that directly or indirectly-cause nucleic acid damage. Anti-cell proliferative activity also can be increased by combining CBP501 with treatments that inhibit cell proliferation whether or not the treatments damage nucleic acid. The invention therefore further provides compositions including CBP501 and a nucleic acid damaging agent, and compositions including CBP501 and an anti-proliferative agent.

As used herein, the terms “abrogate the cell cycle G2 checkpoint,” “disrupt the cell cycle G2 checkpoint,” “impair the cell cycle G2 checkpoint” and grammatical variations thereof, means inhibiting a cell to arrest cell cycle at the G2 checkpoint. A cell in which the cell cycle G2 checkpoint is abrogated exhibits a decrease in the length of time that the cell is in the G2 checkpoint, which can range from absence of G2 checkpoint altogether to a G2 checkpoint having a decrease in duration of minutes, hours, days, weeks or longer under appropriate conditions. Thus, a cell contacted with CBP501 has a G2 checkpoint time shorter in length than the cell normally would have in the absence of the compound. For example, a decrease in the length of G2 checkpoint time would mean that a cell which is in G2 for a certain time, e.g., 4 hours, when contacted with CBP501, is in G2 for less than 4 hours, e.g., 3.5, 3, 2.5, 2, 1 or fewer hours.

As used herein, the term “apoptosis” refers to programmed cell death, and associated changes in cell physiology, e.g., nucleic acid fragmentation, caspase activation, etc., as is understood in the art. The term “catastrophe” means cell death resulting from an error in the mitotic process. In catastrophe, there are fewer features present that are characteristic of apoptosis e.g., caspase activation, chromosome condensation, etc.

As used herein, the terms “peptide,” “polypeptide” and “protein” are used interchangeably and refer to two or more amino acids covalently linked by an amide bond or non-amide equivalent. The peptides can include modifications typically associated with post-translational processing of proteins, for example, cyclization (e.g., disulfide or amide bond), phosphorylation, glycosylation, carboxylation, ubiquitination, myristylation, or lipidation.

As mentioned above, CBP501 has an effect to potentiate cytotoxicity caused by a nucleic acid damaging treatment (e.g., a chemical agent (bleomycins such as bleomycin, platinum-containing drug such as cisplatin etc.) or treatment protocol). Bleomycin-treated cells show a potentiating effect on DNA damage checkpoint signal or foci formation induced by DNA damage, and cisplatin-treated cells show effects to increase the number of G2 phase cells, increase platinum concentration and platinum-DNA adduct in the cell, and enhance checkpoint signals. The enhanced checkpoint signal can be confirmed by examining the variation in the expression levels of checkpoint signal proteins. Specific examples of the checkpoint signal proteins include ATM, H2AX, MAPKAPK2, CHK2, CHK1, Cdc25C and the like, as well as their phosphorylated products (ATM p-Ser1981, γ-H2A, MAPKAPK2 p-Thr222, CHK2-p-Thr68, CHK1 p-Ser317 and Cdc-25 p-Ser216 etc.).

CBP501 can be produced and isolated using any method known in the art. CBP501 can be synthesized, whole or in part, using chemical methods known in the art (see, e.g., Caruthers (1980) Nucleic Acids Res. Symp. Ser. 215-223; Horn (1980) Nucleic Acids Res. Symp. Ser. 225-232; and Banga, A. K., Therapeutic Peptides and Proteins, Formulation, Processing and Delivery Systems (1995) Technomic Publishing Co., Lancaster, Pa.). Peptide synthesis can be performed using various solid-phase techniques (see, e.g., Roberge (1995) Science 269:202; Merrifield (1997) Methods Enzymol. 289:3-13) and automated synthesis may be achieved, e.g., using the ABI 431 A Peptide Synthesizer (Perkin Elmer) in accordance with the manufacturer's instructions.

CBP501 can also be synthesized and expressed as fusion proteins with one or more additional domains linked thereto for producing a more immunogenic peptide, to more readily isolate a recombinantly synthesized peptide, or to identify and isolate antibodies or antibody-expressing B cells. Domains facilitating detection and purification include, for example, metal chelating peptides such as polyhistidine tracts and histidine-tryptophan modules that allow purification on immobilized metals; protein A domains that allow purification on immobilized immunoglobulin; and the domain utilized in the FLAGS extension/affinity purification system (Immunex Corp, Seattle Wash.). The inclusion of a cleavable linker sequence such as Factor Xa or enterokinase (Invitrogen, San Diego Calif.) between a purification domain and the peptide can be used to facilitate peptide purification. For example, an expression vector can include a CBP501-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-14). The histidine residues facilitate detection and purification of the fusion protein while the enterokinase cleavage site provides a means for purifying the peptide from the remainder of the fusion protein. Technology pertaining to vectors encoding fusion proteins and application of fusion proteins is known in the art (see e.g., Kroll (1993) DNA Cell. Biol., 12:441-53).

As used herein, the terms “nucleic acid damaging treatment” and “nucleic acid damaging agent” means any treatment regimen that directly or indirectly damages nucleic acid (e.g., DNA, cDNA, genomic DNA, mRNA, tRNA or rRNA). Specific examples of such agents include alkylating agents, nitrosoureas, anti-metabolites, plant alkaloids, plant extracts and radioisotopes. Specific examples of agents also include nucleic acid damaging drugs, for example, 5-fluorouracil (5-FU), capecitabine, S-1 (Tegafur, 5-chloro-2,4-dihydroxypyridine and oxonic acid), 5-ethynyluracil, arabinosyl cytosine (ara-C), 5-azacytidine (5-AC), 2′,2′-difluoro-2′-deoxycytidine (dFdC), purine antimetabolites (mercaptopurine, azathioprine, thioguanine), gemcitabine hydrochloride (GEMZAR), pentostatin, allopurinol, 2-fluoro-arabinosyl-adenine (2F-ara-A), hydroxyurea, sulfur mustard (bischloroethylsulfide), mechlorethamine, melphalan, chlorambucil, cyclophosphamide, ifosfamide, thiotepa, AZQ, mitomycin C, dianhydrogalactitol, dibromodulcitol, alkyl sulfonate (busulfan), nitrosoureas (BCNU, CCNU, 4-methyl CCNU or ACNU), procarbazine, decarbazine, rebeccamycin, anthracyclins such as doxorubicin (adriamycin; ADR), daunorubicin (CERUBICINE), idarubicin (IDAMYCIN) and epirubicin (ELLENCE), anthracyclin analogues such as mitoxantrone, actinimycin D, non intercalating topoisomerase inhibitors such as epipodophyllotoxins (etoposide=VP16, is teniposide=VM-26), podophylotoxin, bleomycin (Bleo, BLM), pepleomycin, compounds that form adducts with nucleic acid including platinum derivatives (e.g., cisplatin (CDDP), trans analogue of cisplatin, carboplatin, iproplatin, tetraplatin, satraplatin and oxaliplatin), camptothecin, topotecan, irinotecan (CPT-11), and SN-38. Specific examples of nucleic acid damaging treatments include radiation (e.g., ultraviolet (UV), infrared (IR), or alpha-, beta- or gamma-radiation) and environmental shock (e.g., hyperthermia).

As the “nucleic acid damaging treatment” or “nucleic acid damaging agent” to be used in combination with a CBP501 treatment, a “treatment with platinum derivatives” or “platinum derivatives” is preferable, and a “treatment with cisplatin” or “cisplatin” is more preferable.

While the “nucleic acid damaging treatment” or “nucleic acid damaging agent” to be used in combination with a CBP501 treatment is appropriately selected according to the desired point of action, a “treatment with bleomycin” or “bleomycin” can also be used preferably other than the above-mentioned “treatment with cisplatin” or “cisplatin”.

As used herein, the terms “anti-proliferative treatment” and “anti-proliferative agent” means any treatment regimen that directly or indirectly inhibits proliferation of a cell, virus, bacteria or other unicellular or multicellular organism regardless of whether or not the treatment or agent damages nucleic acid. Particular examples of anti-proliferative agents are anti-tumor and anti-viral drugs, which inhibit cell proliferation or virus proliferation or replication. Specific examples include, inter alia, cyclophosphamide, azathioprine, cyclosporine A, prednisolone, melphalan, chlorambucil, mechlorethamine, busulphan, methotrexate, pemetrexed, pemetrexed sodium (ALIMTA), 6-mercaptopurine, thioguanine, cytosine arabinoside, taxol, vinblastine, vincristine, doxorubicin, bleomycin, actinomycin D, mithramycin, carmustine, lomustine, semustine, streptozotocin, hydroxyurea, cisplatin, mitotane, procarbazine, dacarbazine and dibromomannitol. Anti proliferative agents that cause nucleic acid replication errors or inhibit nucleic acid replication such as nucleoside and nucleotide analogues (e.g., AZT or 5-AZC) are also exemplified.

As the “anti-proliferative treatment” or “anti-proliferative agent” to be used in combination with a CBP501 treatment, a “treatment with cisplatin” or “cisplatin” is more preferable.

While the “anti-proliferative treatment” or “anti-proliferative agent” to be used in combination with a CBP501 treatment is appropriately selected according to the desired point of action, a “treatment with bleomycin” or “bleomycin” can also be used preferably other than the above-mentioned “treatment with cisplatin” or “cisplatin”.

Plural kinds of the “nucleic acid damaging treatment” or “nucleic acid damaging agent” may be used in combination with a CBP501 treatment. Similarly, plural kinds of the “anti-proliferative treatment” or “anti-proliferative agent” may be used in combination with a CBP501 treatment. Moreover, (i) CBP501 treatment, (ii) “nucleic acid damaging treatment” or “nucleic acid damaging agent”, and (iii) “anti-proliferative treatment” or “anti-proliferative agent” may be used in combination. For example, a combined use or a combination agent of CBP501, cisplatin and gemcitabine, a combined use or a combination agent of CBP501, cisplatin and pemetrexed and the like can be employed.

CBP501 can also augment the anti-cell proliferative activity of microtubule stabilizing or destabilizing agents such as vinca alkaloids (vinblastine=VLB, vincristin=VCR, vinorelbine=VRLB, vinflunine=VFL), and taxanes (paclitaxel and docetaxel=TAXOTERE). Thus, such agents may be further included in the compositions of the invention and used in the methods of the invention.

Cells that may be treated with CBP501 include any cell whose proliferation it is desired to inhibit or prevent in vitro, ex vivo or in vivo. Particular target cells exhibit a shorter than normal cell cycle G1 checkpoint time or have an impaired cell cycle G1 checkpoint such that the cells exit the G1 checkpoint before enough time has passed to complete nucleic acid repair. Candidate cells therefore include cells that rapidly proliferate whether the cells are normal or abnormal. Specific examples are benign or tumorous, metastatic or non-metastatic cells. Additional candidate cells can be identified by measuring their proliferation rate or the length of time that the cells remain in G1 phase. Candidate cells can also be identified by contacting a test cell with CBP501 alone, or in combination with a nucleic acid damaging treatment, and determining if the contacted cell exhibits decreased proliferation or increased cell death or apoptosis/catastrophe.

CBP501 is therefore useful for inhibiting cell proliferation in vitro, ex vivo and in vivo. As such, subjects having or at risk of having a disorder or physiological condition characterized by abnormal or undesirable or unwanted cell proliferation or cell survival, or abnormal or deficient cell differentiation, can be treated with CBP501 alone or in combination with a treatment that directly or indirectly causes nucleic acid damage or an anti-proliferative treatment.

Thus, in accordance with the invention, there are provided methods for inhibiting cell proliferation, methods for increasing sensitivity of a cell to a nucleic acid damaging agent or treatment and methods for increasing nucleic acid damage to a cell in vitro, ex vivo and in vivo. In one embodiment, a method includes contacting a cell (e.g., a cultured cell or a cell present in a subject) with an amount of CBP501 sufficient to inhibit proliferation of the cell. In another embodiment, a method includes contacting the cell with an amount of CBP501 sufficient to increase sensitivity of the cell to a nucleic acid damaging agent or treatment. In yet another embodiment, a method includes contacting a cell with an amount of CBP501 sufficient to increase nucleic acid damage of the cell. In various aspects, a method further includes contacting the cell with a nucleic acid damaging agent or exposing the cell to a nucleic acid damaging treatment.

Preferably, the treatment with CBP501 is performed simultaneously with or before a nucleic acid damaging treatment (combination treatment). More preferably, the treatment with CBP501 is performed prior to a nucleic acid damaging treatment. For example, CBP501 is administered simultaneously with a nucleic acid damaging agent or, immediately before or 10 min to 6 hr before, preferably immediately before or 10 min to 2 hr before, more preferably immediately before or 10 min to 1 hr before, administration of a nucleic acid damaging agent. A combination treatment of a treatment with CBP501 and a nucleic acid damaging treatment may be performed after a given period after a single administration of CBP501. The given period is generally about 1 to 7 days. In consideration of the condition of the subject of administration and the like, it can be appropriately increased or decreased.

For example, an administration schedule of a combination of a treatment with CBP501 and a treatment with cisplatin is as follows.

### [Schedule 1]

The following step a) and step b) as one cycle is repeated once a week for 3 weeks;
a) step of administering a therapeutically effective amount of CBP501, a prodrug thereof or a pharmaceutically acceptable salt thereof to a mammal by intravenous infusion, and
b) step of administering a therapeutically effective amount of cisplatin to the mammal after completion of step a).

### [Schedule 2]

The following step a) and step b) as one cycle is repeated once every day for 5 consecutive days;
a) step of administering a therapeutically effective amount of CBP501, a prodrug thereof or a pharmaceutically acceptable salt thereof to a mammal by intravenous infusion, and
b) step of administering a therapeutically effective amount of cisplatin to the mammal after completion of step a).

Moreover, for example, an administration schedule of a combination of a treatment with CBP501, a treatment with cisplatin and a treatment with pemetrexed is as follows.

### [Schedule 3]

The following step a)-step c) as one cycle is repeated every 3 weeks;
a) step of administering a therapeutically effective amount of CBP501, a prodrug thereof or a pharmaceutically acceptable salt thereof to a mammal by intravenous infusion,
b) step of administering a therapeutically effective amount of pemetrexed to the mammal after completion of step a), and
c) step of administering a therapeutically effective amount of cisplatin to the mammal after completion of step b).

More specific administration schedule based on Schedule 3 is as follows.

1. CBP501 25 mg/m² will be administered as an i.v. infusion of 1 hr.
2. Pemetrexed 500 mg/m² will be administered as an i.v. infusion over 10 min, immediately after the CBP501 infusion.
3. Cisplatin 75 mg/m² will be administered as a 1 hr i.v. infusion immediately after the pemetrexed infusion.

In the present invention, depending on the desired action of CBP501 or the kind of the “nucleic acid damaging treatment” to be used in combination with a CBP501 treatment, a treatment with CBP501 can be performed after a nucleic acid damaging treatment. Example thereof is a combined use of oxaliplatin or carboplatin.

Further provided are methods of treating a cell proliferative disorder or differentiative disorder in a subject, including conditions characterized by undesirable or unwanted cell proliferation or cell survival, conditions characterized by deficient or aberrant apoptosis, conditions characterized by aberrant or deficient cell survival, as well as conditions characterized by aberrant or deficient cell differentiation. In one embodiment, a method includes administering to a subject having or at risk of having a cell proliferative disorder, an amount of CBP501 effective to treat the cell proliferative disorder. In one aspect, the amount is sufficient to improve the subject's condition. In particular aspects, the improvement includes, in at least a portion of the target cells (e.g., abnormally proliferating cells), decreased cell proliferation, decreased numbers of cells, inhibiting increases in the number of cells, increased apoptosis, or decreased survival. In yet another aspect, the subject is administered CBP501 prior to, contemporaneously with, or after administering (or performing) a treatment that inhibits cell proliferation. Preferably, the subject is administered CBP501 prior to, or contemporaneously with administering (or performing) a treatment that inhibits cell proliferation. More preferably, the subject is administered CBP501 prior to administering a (or performing) treatment that inhibits cell proliferation. In additional particular aspects, at least a part of the cells of the cell proliferative disorder are located in blood, breast, lung, thyroid, head or neck, brain, lymph, gastrointestinal tract, genito-urinary tract, kidney, pancreas, liver, bone, muscle, or skin.

In another embodiment, a method includes administering an amount of CBP501 to the subject to treat a solid tumor. In yet another embodiment, a method includes administering an amount of CBP501 to the subject to treat a liquid tumor in various aspects, the subject having the tumor is administered with CBP501 prior to, contemporaneously with, or after another anti-tumor therapy. Preferably, the subject is administered CBP501 prior to, or contemporaneously with another anti-tumor therapy. More preferably, the subject is administered CBP501 prior to another anti-tumor therapy.

As used herein, the terms “proliferative disorder” and “proliferative condition” mean any pathological or non-pathological physiological condition characterized by aberrant or undesirable proliferation (e.g., of a cell, virus, bacteria, fungus, etc.). The terms “cell proliferative disorder” and “cell proliferative condition” mean any pathological or non-pathological physiological condition characterized by aberrant or undesirable cell proliferation, as well as including conditions characterized by undesirable or unwanted cell proliferation or cell survival (e.g., due to deficient apoptosis), conditions characterized by deficient or aberrant or deficient apoptosis, as well as conditions characterized by aberrant or undesirable or unwanted cell survival. The term “differentiative disorder” means any pathological or non-pathological physiological condition characterized by aberrant or deficient differentiation.

Proliferative or differentiative disorders amenable to treatment include diseases and non-pathological physiological conditions, both benign and neoplastic, characterized by abnormal or undesirable cell numbers, cell growth or cell survival. Such disorders or conditions may therefore constitute a disease state and include all types of cancerous growths or oncogenic processes, metastatic tissues or malignantly transformed cells, tissues, or organs, or may be non-pathologic, i.e., a deviation from normal but which is not typically associated with disease. A specific example of a non-pathologic condition that may be treated in accordance with the invention is tissue re-growth from wound repair that results in scarring.

Cells comprising the proliferative or differentiative disorder may be aggregated in a cell mass or be dispersed.

The term “solid tumor” refers to neoplasias or metastases that typically aggregate together and form a mass. Particular examples include visceral tumors such as gastric or colon cancer, hepatomas, venal carcinomas, lung and brain tumors/cancers. A “liquid tumor” refers to neoplasias of the haematopoietic system, such as lymphomas, myelomas and leukemias, or neoplasias that are diffuse in nature, as they do not typically form a solid mass. Particular examples of leukemias include acute and chronic lymphoblastic, myeloblastic and multiple myeloma.

Such disorders include neoplasms or cancers, which can affect virtually any cell or tissue type, e.g., carcinoma, sarcoma, melanoma, metastatic disorders or haematopoietic neoplastic disorders. A metastatic tumor can arise from a multitude of primary tumor types, including but not limited to breast, lung, thyroid, head and neck, brain, lymphoid, gastrointestinal (mouth, esophagus, stomach, small intestine, colon, rectum), genito-urinary tract (uterus, ovary, cervix, bladder, testicle, penis, prostate), kidney, pancreas, liver, bone, muscle, skin, etc.

Carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from the cervix, lung, prostate, breast, head and neck, colon, liver and ovary. The term also includes carcinosarcomas, e.g., which include malignant tumors composed of carcinomatous and sarcomatous tissues. Adenocarcinoma includes a carcinoma of a glandular tissue, or in which the tumor forms a gland like structure.

Sarcomas refer to malignant tumors of mesenchymal cell origin. Exemplary sarcomas include for example, lymphosarcoma, liposarcoma, osteosarcoma, and fibrosarcoma.

As used herein, the term “haematopoietic proliferative disorder” means a disease involving hyperplastic/neoplastic cells of haematopoietic origin, e.g., arising from myeloid, lymphoid or erythroid lineages, or precursor cells thereof. Typically, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional malignant lymphomas include, but are not limited to, non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

Accordingly, in the present invention, diseases to be treated may be selected from various cancers, especially, breast cancer (e.g., invasive ductal breast cancer, non-invasive ductal breast cancer, inflammatory breast cancer etc.), prostate cancer (e.g., hormone dependent prostate cancer, hormonal-independent prostate cancer etc.), pancreas cancer (e.g., pancreatic duct cancer etc.), gastric cancer (e.g., papillary adenocarcinoma, mucous adenocarcinoma, adenosquamous carcinoma etc.), lung cancer (e.g., non-small cell lung cancer, small cell lung cancer, malignant mesothelioma (pleural mesothelioma, peritoneal mesotheliomas, pericardial mesothelioma etc.) etc.), colon cancer (e.g., gastrointestinal stromal tumor etc.), rectal cancer (e.g., gastrointestinal stromal tumor etc.), large bowel cancer (e.g., familial large bowel cancer, hereditary nonpolyposis colon cancer, gastrointestinal stromal tumor etc.), small intestinal cancer (e.g., non Hodgkin's lymphoma, gastrointestinal stromal tumor etc.), esophageal cancer, duodenal cancer, lingual cancer, pharyngeal cancer (e.g., nasopharyngeal cancer, mesopharyngeal cancer, hypopharynx cancer etc.), salivary gland cancer, cerebral tumor (e.g., pineal astrocytoma, pilocytic astrocytoma, diffuse astrocytoma, anaplastic astrocytoma etc.), schwanoma, liver cancer (e.g., primary liver cancer, extrahepatic bile duct cancer etc.), kidney cancer (e.g., renal cell cancer, transitional cell cancer of the ureter and the renal pelvis etc.), bile duct cancer, endometrial cancer, cervical cancer, ovarian cancer (e.g., epithelial ovarian cancer, extragonadal germ cell tumor, ovarian germ cell tumor, ovarian low-malignant potential tumor etc.), bladder cancer, urethral cancer, skin cancer (e.g., intraocular melanoma, Merkel cell carcinoma etc.), angioma, malignant lymphoma, malignant melanoma, thyroid cancer (e.g., medullary thyroid cancer etc.), parathyroid cancer, nasal cancer, paranasal cancer, auditory organ cancer, carcinoma of oral floor, laryngeal cancer, unknown primary cancer, parotid cancer, submandibular cancer, bone tumor (e.g., osteosarcoma, Ewing's tumor, uterine sarcoma, soft tissue sarcoma etc.), angiofibroma, retinal sarcoma, penile cancer, testicular tumor, pediatric solid cancer (e.g., Wilms tumor, pediatric kidney tumor etc.), Kaposi's sarcoma, Kaposi's sarcoma resulted from AIDS, tumor of maxillary sinus, fibrous histiocytoma, leiomyosarcoma, rhabdomyosarcoma, multiple myeloma, and leukemia (e.g., acute myelocytic leukemia, acute lymphoblastic leukemia etc.) etc.

Treatments for use in combination with CBP501 include any anti-proliferative, nucleic acid damaging or anti-tumor treatment as disclosed herein or known in the art. For example, an anti-cell proliferative or anti-tumor treatment may comprise radiation treatment or surgical resection optionally in combination with drug treatment. The treatment may comprise administration of a chemical substance, such as a radioisotope, a drug, such as a chemotherapeutic agent, or genetic therapy, such as an anti-oncogene (e.g., Rb, DCC, p53, etc.), a dominant negative oncogene or an antisense to an oncogene. The compounds can be administered prior to, contemporaneously with or following other treatment protocols. For example, a candidate subject for anti-cell proliferative therapy (e.g., radiation therapy, chemotherapy, gene therapy, surgical resection, etc.) can be administered CBP501 prior to initiating the anti-cell proliferative therapy. Thus, prophylactic treatment methods are provided.

Furthermore, treatments for use in combination with CBP501 include any prophylactic anti-allergy treatment as disclosed herein or known in the art. For example, an anti-allergy treatment may comprise administration of a chemical substance, such as an antiallergic agent (e.g., denocorticotropic hormone (e.g., dexamethasone etc.) antihistamine drug (e.g., diphenhydramine, loratadine, etc.), etc.). The antiallergic agent can be administered prior to, contemporaneously with or following administration of CBP501.

The term “subject” refers to animals, typically mammalian animals, such as primates (humans, apes, gibbons, chimpanzees, orangutans, macaques), domestic animals (dogs and cats), farm animals (horses, cattle, goats, sheep, pigs) and experimental animals (mouse, rat, rabbit, guinea pig). Subjects include animal disease models (e.g., tumor bearing mice).

Subjects appropriate for treatment include those currently undergoing or are candidates for treatment for a proliferative or differentiative disorder (e.g., anti-tumor therapy). Additional candidate subjects include, for example, subjects at risk of developing a cell proliferative disorder. The invention methods are therefore applicable to treating a subject who is at risk of developing a cell proliferative disorder but who has not yet exhibited overt symptoms of the disorder. At risk subjects can be identified as having a genetic predisposition or family history to developing a cell proliferative disorder. For example, subjects having an activated oncogene or having a mutation or deletion of a tumor suppressor gene are candidate subjects. At risk subjects can therefore be identified using routine genetic screening for the presence of the genetic lesion, or inquiry into the subjects' family history to establish that they are at risk of the disorder. A particular example of an at risk subject would be one with a family history or other genetic characteristic indicating predisposition to a cancer in which the neoplastic or drug-resistant neoplastic cells express CD40. A particular specific example of a genetic disease is retinoblastoma, which is caused by a defect in the Rb tumor suppressor gene.

Amounts administered are typically in an “effective amount” or “sufficient amount” that is an amount sufficient to produce the desired affect. Effective amounts therefore include one or more of: decreasing cell proliferation, decreasing numbers of cells, inhibiting increased proliferation, inhibiting increased numbers of cells, increasing apoptosis, or decreasing survival, of at least a portion of the cells comprising the proliferating cells (e.g., at least some of the target cells). Thus, for example, where it is desired to inhibit cell proliferation, an effective amount will be an amount that detectably decreases cell proliferation or numbers of proliferating cells, or increases cell apoptosis or decreases cell survival. The amount can therefore be sufficient to reduce target cell numbers, stabilize target cell numbers or inhibit increases in target cell numbers. For example, where the disorder comprises a solid tumor, reducing tumor size, stabilizing tumor size, or preventing further growth of the tumor, of at least a portion of the tumor (e.g. inhibiting growth of 5-10% of the cells, or 10-20% or more of the cells comprising the tumor mass) is a satisfactory clinical endpoint. Where the disorder comprises a liquid tumor, reducing numbers of tumor cells, stabilizing tumor cell numbers or inhibiting further increases in tumor cell numbers, of at least a subpopulation of the tumor cells (e.g. inhibiting growth of 5-10% of the cells, or 10-20% or more of the cells) is a satisfactory clinical endpoint.

In addition, amounts considered effective can prevent or inhibit progression of the condition or disorder. For example, certain tumors as they progress become increasingly aggressive, including progressing to metastatic forms. Thus, amounts also considered effective would result in reducing or preventing the tumors from becoming increasingly aggressive or from metastasizing. Accordingly, inhibiting or preventing a worsening of the disorder or condition, i.e., stabilizing the condition is an additional satisfactory clinical endpoint.

Examination of a biological sample containing a liquid tumor (e.g., blood or a tissue sample), can establish whether tumor cell mass or numbers have been reduced, or inhibition of tumor cell proliferation has occurred. For a solid tumor, invasive and non-invasive imaging methods can ascertain a reduction in tumor size, or inhibiting increases in the tumor size. Decreasing counts of receptor of a receptor positive tumor, can be used to assess reduction or inhibition of tumor cell proliferation. Amounts of hormone of a hormone producing tumor, e.g., breast, testicular, or ovarian cancers, can be used to assess a reduction or inhibition of proliferation of the tumor.

Effective amounts can also objectively or subjectively reduce or decrease the severity or frequency of symptoms associated with the disorder or condition. For example, an amount of CBP501 that reduces pain, nausea or other discomfort, or increases appetite or subjective well being is a satisfactory clinical endpoint.

Effective amounts also include a reduction of the amount (e.g., dosage) or frequency of treatment with another protocol, which is considered a satisfactory clinical endpoint. For example, a cancer patient treated with CBP501 may require less nucleic acid damaging treatment in order to inhibit cancer cell proliferation. In this example, an effective amount would include an amount that reduces the dosage frequency or amount of a nucleic acid damaging agent that the subject is administered in comparison to the dosage frequency or amount administered without treatment with CBP501.

Methods of the invention that lead to an improvement in the subject's condition or a therapeutic benefit may be relatively short in duration, e.g., the improvement may last several hours, days or weeks, or extend over a longer period of time, e.g., months or years. An effective amount need not be a complete ablation of any or all symptoms of the condition or disorder. Thus, a satisfactory clinical endpoint for an effective amount is achieved when there is a subjective or objective improvement in the subjects' condition as determined using any of the foregoing criteria or other criteria known in the art appropriate for determining the status of the disorder or condition, over a short or long period of time. An amount effective to provide one or more beneficial effects, as described herein or known in the art, is referred to as an “improvement” of the subject's condition or “therapeutic benefit” to the subject.

An effective amount of CBP501 can be determined based upon animal studies or optionally in human clinical trials. The skilled artisan will appreciate the various factors that may influence the dosage and timing required to treat a particular subject including, for example, the general health, age, or gender of the subject, the severity or stage of the disorder or condition, previous treatments, susceptibility to undesirable side effects, clinical outcome desired and the presence of other disorders or conditions. Such factors may influence the dosage and timing required to provide an amount sufficient for therapeutic benefit. The dosage regimen also takes into consideration the pharmacokinetics, i.e., the pharmaceutical composition's rate of absorption, bioavailability, metabolism, and clearance (see, e.g., Egleton (1997) “Bioavailability and transport of peptides and peptide drugs into the brain” Peptides 18:1431-1439; and Langer (1990) Science 249:1527-1533). In addition, doses or treatment protocols may be specifically tailored to the subject or modified based on pharmacogenomic data.

CBP501 can therefore be administered alone or as a pharmaceutical composition, systemically, regionally (e.g., directed towards an organ or tissue, e.g., by injection into the portal vein for treating a cell proliferative disorder of the liver), or locally (e.g., directly into a tumor mass), in accordance with any protocol or route that achieves the desired effect. The =501 and pharmaceutical compositions thereof can be administered as a single or multiple dose each day (e.g., at a low dose), or intermittently (e.g., every other day, once a week, etc. at a higher dose). The CBP501 and pharmaceutical compositions thereof can be administered via inhalation (e.g., intra-tracheal), orally, intravenously, is intraarterially, intravascularly, intrathecally, intraperitoneally, intramuscularly, subcutaneously, intracavity, transdermally (e.g., topical), transmucosally (e.g., buccal, bladder, vaginal, uterine, rectal, or nasal), by multiple administrations, sustained release (e.g., gradual perfusion over time) or a single bolus. Implantable devices, including microfabricated devices, for administering drugs are well known and are also applicable for delivering compounds of the invention to a subject.

CBP501 administered intravenously (IV) would be at about 1.0 mg/hr to about 75 mg/hr over several hours (typically 1, 3, or 6 hours), which can be repeated for one or more weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF).

As mentioned above, CBP501 can be used in combination with a nucleic acid damaging agent and/or an anti-cell proliferative agent, and the combined use is preferable. When in combined use, CBP501 can be administered according to the above-mentioned CBP501 single administration (single-agent). Since a more superior effect can be expected by the combined use, the dose, dosing period, administration frequency and the like of CBP501 can be reduced.

The nucleic acid damaging agent and/or the anti-cell proliferative agent to be combined with CBP501 can be administered according to the clinically-employed administration standard, respectively. Since a more superior effect can be expected by the combined use, the dose, dosing period, administration frequency and the like of the nucleic acid damaging agent and/or the anti-cell proliferative agent can be reduced.

For example, the following administration schedule is employed.

### [Schedule 1]

The following step a) and step b) as one cycle is repeated once a week for 3 weeks;
a) step of administering a therapeutically effective amount of CBP501, a prodrug thereof or a pharmaceutically acceptable salt thereof to a mammal by intravenous infusion, and
b) step of administering a therapeutically effective amount of cisplatin to the mammal after completion of step a).

### [Schedule 2]

The following step a) and step b) as one cycle is repeated once every day for 5 consecutive days;
a) step of administering a therapeutically effective amount of CBP501, a prodrug thereof or a pharmaceutically acceptable salt thereof to a mammal by intravenous infusion, and
b) step of administering a therapeutically effective amount of cisplatin to the mammal after completion of step a).

Moreover, for example, the protocol of a combination of a treatment with CBP501, a treatment with cisplatin and a treatment with pemetrexed is as follows.

### [Schedule 3]

The following step a)-step c) as one cycle is repeated every 3 weeks;
a) step of administering a therapeutically effective amount of CBP501, a prodrug thereof or a pharmaceutically acceptable salt thereof to a mammal by intravenous infusion,
b) step of administering a therapeutically effective amount of pemetrexed to the mammal after completion of step a), and
c) step of administering a therapeutically effective amount of cisplatin to the mammal after completion of step b).

More specific administration schedule based on Schedule 3 is as follows.

1. CBP501 25 mg/m² will be administered as an i.v. infusion of 1 hr.
2. Pemetrexed 500 mg/m² will be administered as an i.v. infusion over 10 min, immediately after the CBP501 infusion.
3. Cisplatin 75 mg/m² will be administered as a 1 hr i.v. infusion immediately after the pemetrexed infusion.

The invention therefore further provides pharmaceutical compositions. Such pharmaceutical compositions are useful for administration to a subject in vivo or ex vivo, and for treating a subject with CBP501, for example.

As used herein, a “pharmaceutical composition” or “pharmaceutical formulation” means a mixture of CBP501 (including a physiologically acceptable salt or prodrug thereof), with one or more additional chemical components, such as pharmaceutically acceptable or physiologically acceptable carriers and excipients. The terms “pharmaceutically acceptable” and “physiologically acceptable” carriers and excipients include solvents (aqueous or non-aqueous), solutions, emulsions, dispersion media, coatings, isotonic and absorption promoting or delaying agents, compatible with pharmaceutical administration. A “pharmaceutical composition” or “pharmaceutical formulation” therefore refers to a composition suitable for administration to a subject. A “pharmaceutically acceptable salt” means a compound in a charged form together with a counter-ion.

As a physiologically acceptable salt of CBP501, for example, a salt with inorganic base, a salt with organic base, a salt with inorganic acid, a salt with organic acid, a salt with basic or acidic amino acid and the like can be mentioned. Such salt can be produced by a method known per se (e.g., acetate salt can be produced by a step of liquid chromatography using acetate-containing solvent, see Examples for more detail).

Preferable examples of salts with inorganic base include alkali metal salt such as sodium salt, potassium salt and the like, alkaline earth metal salt such as calcium salt, magnesium salt and the like, and aluminum salt, ammonium salt and the like.

Preferable examples of salts with organic base include salts with trimethylamine, triethylamine, pyridine, picoline, ethanolamine, diethanolamine, triethanolamine, tromethamine[tris(hydroxymethyl)aminomethane], tert-butylamine, cyclohexylamine, benzylamine, dicyclohexylamine, N,N′-dibenzylethylenediamine and the like.

Preferable examples of salts with inorganic acid include salts with hydrochloric acid, hydrobromic acid, nitric acid, sulfuric acid, phosphoric acid and the like.

Preferable examples of salts with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of salts with basic amino acid include salts with arginine, lysine, ornithine and the like.

Preferable examples of salts with acidic amino acid include salts with aspartic acid, glutamic acid and the like.

Preferred is a salt with an organic acid such as acetic acid and the like.

The number of acetic acid attached to CBP501 can vary, and 4 or 5 acetic acids are preferably attached. Alternatively, a mixture of CBP501 acetate salts having different number of acetic acids attached thereto (for example, mixture of 4 acetate and 5 acetate etc.) may be used.

As used herein, a “prodrug” is a compound that is metabolized, converted or modified to an active form, e.g., CBP501 itself, in vivo. Prodrugs are often useful because they may be easier to administer than the parent drug or exhibit increased bioavailability or solubility as compared to the parent drug. A particular non-limiting example of a prodrug is a polypeptide which is bonded through an amino- or a carboxy-terminal group to CBP501. The polypeptide hydrolyzes or is metabolized in vivo to release the CBP501. The invention compounds and methods therefore include prodrugs of CBP501 that are metabolized, converted or modified in vivo to an active form of CBP501.

The composition may contain, as an active ingredient in addition to CBP501, nucleic acid damaging agent and/or anti-cell proliferative agent. As the nucleic acid damaging agent, those exemplified above can be used. As the anti-cell proliferative agent, those exemplified above can be used (hereinafter a composition containing, in addition to CBP501, nucleic acid damaging agent and/or anti-cell proliferative agent is also referred to as a combination agent of the present invention).

The amount of CBP501, and the amount(s) of nucleic acid damaging agent and/or anti-cell proliferative agent in the combination agent of the present invention can be appropriately determined in consideration of their amounts for singly use. Since a more superior treatment effect can be expected by the effect afforded by CBP501 to potentiate cytotoxicity of nucleic acid damaging agents, and the effect afforded by the anti-cell proliferative agent to potentiate a cell proliferation suppressive action, the amounts can be set lower than those for singly use.

For example, a combination agent of CBP501 (25 mg) and cisplatin (75 mg), a combination agent of CBP501 (25 mg), cisplatin (75 mg) and pemetrexed (500 mg), both per body surface area (/m²), and the like can be mentioned.

Pharmaceutical compositions can be formulated to be compatible with a particular route of administration, systemic or local. Thus, pharmaceutical compositions include carriers, diluents, or excipients suitable for administration by various routes.

Formulations or enteral (oral) administration can be contained in a tablet (coated or uncoated), capsule (hard or soft), microsphere, emulsion, powder, granule, crystal, suspension, syrup or elixir. Conventional nontoxic solid carriers which include, for example, pharmaceutical grades of mannitol, lactose, starch, magnesium stearate, sodium saccharin, talcum, cellulose, glucose, sucrose, magnesium carbonate, can be used to prepare solid formulations. Supplementary active compounds (e.g., preservatives, antibacterial, antiviral and antifungal agents) can also be incorporated into the formulations. A liquid formulation can also be used for enteral administration. The carrier can be selected from various oils including petroleum, animal, vegetable or synthetic, for example, peanut oil, soybean oil, mineral oil, sesame oil. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Pharmaceutical compositions for enteral, parenteral, or transmucosal delivery include, for example, water, saline, phosphate buffered saline, Hank's solution, Ringer's solution, dextrose/saline, and glucose solutions. The formulations can contain auxiliary substances to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. Additional parenteral formulations and methods are described in Bai (1997) J. Neuroimmunol. 80:65-75; Warren (1997) J. Neurol. Sci. 152:31-38; and Tonegawa (1997) J. Exp. Med. 186:507-515. The parenteral preparation can be enclosed in ampoules, disposable syringes or multiple dose vials made of glass or plastic.

Pharmaceutical compositions for intradermal or subcutaneous administration can include a sterile diluent, such as water, saline solution, fixed oils, polyethylene glycols, glycerine, propylene glycol or other synthetic solvents; antibacterial agents such as benzyl alcohol or methyl parabens; antioxidants such as ascorbic acid, glutathione or sodium bisulfite; chelating agents such as ethylenediaminetetraacetic acid; buffers such as acetates, citrates or phosphates and agents for the adjustment of tonicity such as sodium chloride or dextrose.

Pharmaceutical compositions for injection include aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyethyleneglycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, sodium chloride may be included in the composition. The resulting solutions can be packaged for use as is, or lyophilized, the lyophilized preparation can later be combined with a sterile solution prior to administration.

Pharmaceutically acceptable carriers can contain a compound that stabilizes, increases or delays absorption or clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (see, e.g., Fix (1996) Pharm Res. 13:1760-1764; Samanen (1996) J. Pharm. Pharmacol. 48:119-135; and U.S. Pat. No. 5,391,377, describing lipid compositions for oral delivery of therapeutic agents).

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be through nasal sprays or suppositories (see, e.g., Sayani (1996) “Systemic delivery of peptides and proteins across absorptive mucosae” Crit. Rev. Ther. Drug Carrier Syst. 13:85-184). For transdermal administration, the active compound can be formulated into ointments, salves, gels, or creams as generally known in the art. Transdermal delivery systems can also be achieved using patches.

For inhalation delivery, the pharmaceutical formulation can be administered in the form of an aerosol or mist. For aerosol administration, the formulation can be supplied in finely divided form along with a surfactant and propellant. In another embodiment, the device for delivering the formulation to respiratory tissue is in which the formulation vaporizes. Other delivery systems known in the art include dry powder aerosols, liquid delivery systems, inhalers, air jet nebulizers and propellant systems (see, e.g., Patton (1998) Biotechniques 16:141-143; Dura Pharmaceuticals, San Diego, Calif.; Aradigm, Hayward, Calif.; Aerogen, Santa Clara, Calif.; and Inhale Therapeutic Systems, San Carlos, Calif.).

Biodegradable, biocompatible polymers can be used, such as ethylene vinyl acetate, polyanhydrides, polyglycolic acid, collagen, polyorthoesters, and polylactic acid. Methods for preparation of such formulations are known to those skilled in the art. The materials can also be obtained commercially from Alza Corporation and Nova Pharmaceuticals, Inc. Liposomal suspensions (including liposomes targeted to cells or tissues using antibodies or viral coat proteins) can also be used as pharmaceutically acceptable carriers. These can be prepared according to methods known in the art, for example, as described in U.S. Pat. Nos. 4,235,871; 4,501,728; 4,522,811; 4,837,028; 6,110,490; 6,096,716; 5,283,185; 5,279,833; Akimaru (1995) Cytokines Mol. Ther. 1:197-210; Alving (1995) Immunol. Rev. 145:5-31; and Szoka (1980) Ann. Rev. Biophys. Bioeng. 9:467). Biodegradable microspheres or capsules or other biodegradable polymer configurations capable of sustained delivery of small molecules including peptides are known in the art (see, e.g., Putney (1998) Nat. Biotechnol. 16:153-157). CBP501 can be incorporated within micelles (see, e.g., Suntres (1994) J. Pharm. Pharmacol. 46:23-28; Woodle (1992) Pharm. Res. 9:260-265). CBP501 can be attached to the surface of the lipid monolayer or bilayer. For example, CBP501 can be attached to hydrazide-PEG-(distearoylphosphatidyl) ethanolamine-containing liposomes (see, e.g., Zalipsky (1995) Bioconjug. Chem. 6:705-708). Alternatively, any form of lipid membrane, such as a planar lipid membrane or the cell membrane of an intact cell, e.g., a red blood cell, can be used. Liposomal and lipid-containing formulations can be delivered by any means, including, for example, intravenous, transdermal (see, e.g., Vutla (1996) J. Pharm. Sci. 85:5-8), transmucosal, or oral administration.

A pharmaceutically acceptable formulation can incorporate about 1% to 99.9% of active ingredient (e.g., CBP501). The pharmaceutical compositions can be sterilized by conventional, well-known sterilization techniques, or can be sterile filtered.

Additional pharmaceutical formulations and delivery systems are known in the art and are applicable in the methods and compositions of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms, Technonic Publishing Co., Inc., Lancaster, Pa., (1993); and Poznansky et al., Drug Delivery Systems, R. L. Juliano, ed., Oxford, N.Y. (1980), pp. 253-315). The pharmaceutical formulations can be packaged in unit dosage form for ease of administration and uniformity of dosage. “Unit dosage form” as used herein refers to physically discrete unitary dosages for administration to the subject to be treated; each unit contains a predetermined quantity of compound that produces a desired effect in combination with a pharmaceutical carrier or excipient.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All publications, patents and other references cited herein are incorporated by reference in their entirety. In case of conflict, the present specification, including definitions, will control.

As used herein, the singular forms “a”, “and,” “the” and “is” include plural referents unless the context clearly indicates otherwise. Thus, for example, reference to a “compound” includes a plurality of compounds and reference to “a residue” or an “amino acid” includes reference to one or more residues and amino acids.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

### Production of H-(D)p-Benzoylphenylalanyl-(D)seryl-(D)tryptophyl-(D)seryl-(D)pentafluorophenylalanyl-(D)cyclohexylalanyl-(D)arginyl-(D)arginyl-(D)arginyl-(D)glutaminyl-(D)arginyl-(D)arginine acetate salt [i.e., H-(d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg)-OH acetate salt; (CBP501 acetate salt)] (SEQ ID NO: 1)

### Property of CBP501

Molecular formula: C₈₆H₁₂₂N₂₉O₁₇F₅
Average Molecular Weight: 1929.1 (anhydrous free base)

### General Properties:

CBP501 acetate salt (CBP501-Drug Substance) is a white to off-white amorphous powder.

CBP501 forms a clear and colorless solution when dissolved at 50 mg/mL in water.

The production of CBP501 is performed through classical chemical reactions. All methods used throughout the production of the peptide are based on very well documented organic reactions used for many years in peptide chemistry.

These aspects of peptide chemistry related to synthesis and analysis are, for instance, well documented in the well-known series: <<The Peptides, Analysis, Synthesis, Biology>>, vol. 1 to 9, S. Udenfried, J. Meienhofer, 1977 to 1987, Academic Press, New York.

A schematic representation of the overall process is provided hereafter, followed by detailed flow charts.

### Process Flow Diagram

TABLE 1
Solid Phase Peptide Assembly

H-(d-Bpa)-(d-Ser) (tBu)-(d-Trp) (Boc)-(d-Ser)
(tBu)-(d-Phe-2,3,4,5,6-F)-(d-Cha)-(d-Arg) (Pbf)-
(d-Arg) (Pbf)-(d-Arg) (Pbf)-(d-Gln) (Trt)-(d-Arg)
(Pbf)-(d-Arg) (Pbf)-Wang Resin
Protected peptide-resin

↓
Deprotection and Cleavage

(SEQ ID NO: 1)

H-(d-Bpa)-(d-Ser)-(d-Trp)-(d-Ser)-(d-Phe-
2,3,4,5,G-F)-(d-Cha)-(d-Arg)-(d-Arg)-(d-Arg)-(d-
Gln)-(d-Arg)-(d-Arg)-OH
Crude CBP501

↓
Purification and Isolation

(SEQ ID NO: 1)

H-(d-Bpa)-(d-Ser)-(d-Trp)-(d-Ser)-(d-Phe-
2,3,4,5,G-F)-(d-Cha)-(d-Arg)-(d-Arg)-(d-Arg)-(d-
Gln)-(d-Arg)-(d-Arg)-OH
CBP501-Drug Substance

### Detailed Flow Charts

TABLE 2

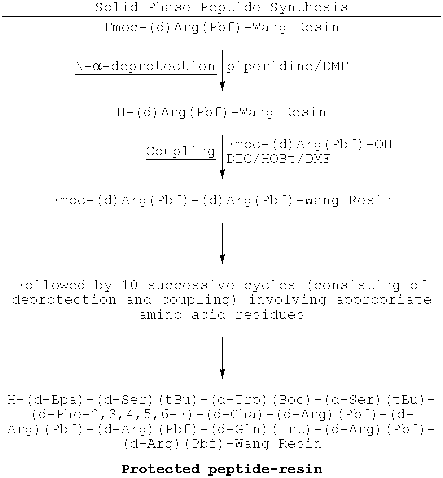

TABLE 3

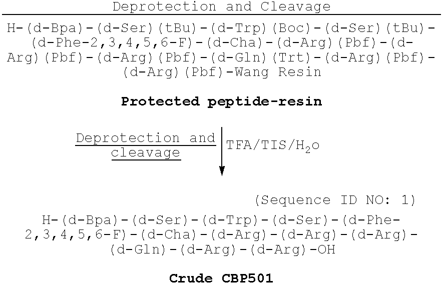

TABLE 4

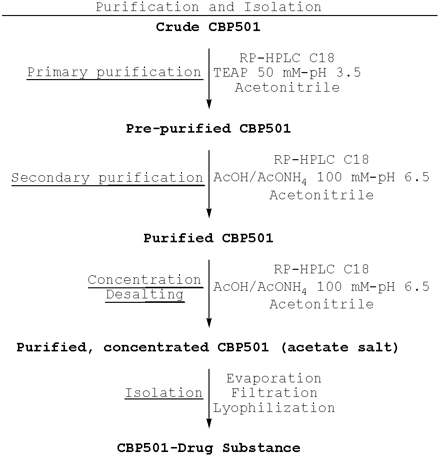

The synthesis of the protected peptide was carried out by stepwise solid phase method on a semi-automated larger scale solid phase peptide synthesizer using a polystyrene 1% divinylbenzene resin carrying N-α-fluorenylmethyloxycarbonyl-arginyl(Pbf), as solid support.

The side chain protecting groups of the amino acid moieties and the peptide-resin bond were cleaved with a mixture of TFA, TIS and processed water H1, leading to crude CBP501.

The crude CBP501 was submitted to three preparative reverse phase HPLC steps for purification, concentration and desalting, to allow for the obtaining of the purified CBP501 under the appropriate salt form being the acetate salt.

The pure CBP501 solution was evaporated under reduced pressure and freeze-dried in order to remove residual acetonitrile. CBP501 powder was dissolved in processed water H2, filtered through a 0.2 μm filter and lyophilized.

All the protected amino acids used as starting materials during the synthesis of CBP501 were controlled and released according to their specifications including at least appearance, identification by HPLC, purity by HPLC, water contents, specific optical rotation, enantiomeric purity and assay by potentiometric titration or nitrogen content. Any starting materials are commercially available or can be produced in accordance with the known methods per se.

All the solvents and reagent used during the synthesis is of CBP501-drug substance were controlled and/or released according to specifications prior their usage in order to ensure the consistency of their quality. A list of the solvents and reagents used is given hereafter.

### Synthesis of Crude CBP501

Dichloromethane, technical grade (DCM)

Methanol, technical grade

Trifluoroacetic acid (TFA)

1-Hydroxybenzotriazole (HOBt)

Diisopropylether (DIPE)

Diisopropylethylamine (DIPEA)

Piperidine

Processed water H1

N,N′-Diisopropylcarbodiimide (DIC)

(Benzotriazol-1-yloxy)-Tripyrrolidinophosphonium-hexafluorophosphate (PyBOP)

Nitrogen (liquid)

N,N′-Dimethylformamide-peptide synthesis grade (DMF)

Triisopropylsilane (TIS)

### Purification and Isolation of CBP501

Acetic acid, glacial

Triethylamine

Phosphoric acid

Ammonium acetate

Ammonia

Acetone (technical grade)

Acetonitrile

Processed water H2

Isopropanol

Nitrogen (liquid)

### Materials Used During the Purification

CBP501 was purified by successive preparative chromatographic HPLC using a reverse phase stationary phase: C18 silica gel (SC-009). This phase was accepted on basis of the supplier's certificate.

Mass determination by ES-MS, Sequencing by MS-MS, 2D-NMR, Amino acid relative composition by AAA method, enantiomeric purity by GC-MS and Acetic acid content by HPLC of obtained CBP501-Drug Substance (CBP501 acetate salt) were performed. Expected values and those actually measured were compared.

TABLE 5

| Test | Expected | Found |
|---|---|---|
| Mass deter- | M_{monoisotopic} = | 1928.0 |
| mination by | 1928.0 ± 1.0 | |
| ES-MS | | |
| Sequencing | H-(d-Bpa) (d-Ser) (d- | A part of the |
| by MS-MS | Trp) (d-Ser) (d-Phe- | sequence is |
| | 2,3,4,5,6-F) (d-Cha) (d- | confirmed |
| 2D-NMR | Arg) (d-Arg) (d-Arg) (d- | Conform with |
| | Gln) (d-Arg) (d-Arg)-OH | the expected |
| | (SEQ ID NO: 1) | sequence |
| Amino acid | | |
| relative | | |
| composition | | |
| by AAA | | |
| Bpa | 1 | (1) |
| Ser | 2 | 1.8 |
| Glu | 1 | 1.01 |
| PFPh | 1 | 1.03 |
| Cha | 1 | 0.99 |
| Trp | 1 | Identified (2) |
| Arg | 5 | 4.97 |
| Enantiomer- | | |
| ic purity | | |
| by GC-MS | | |
| Ser | d-Ser | <0.1% 1-Ser |
| PFPh | d-PFPh | 0.17% 1-PFPh |
| Glu | d-Glu | 0.22% 1-Glu |
| Arg | d-Arg | 0.11% 1-Arg |
| Trp | d-Trp | 0.18% 1-Trp |
| Cha | d-Cha | <0.1% 1-Cha |
| Bpa | d-Bpa | 0.23% 1-Bpa |
| Acetic acid | ≦18% | 12.6% |
| content by | | |
| HPLC | | |

(1) Bpa is not detected by the AAA method.
(2) Trp may not be quantified due to its instability in the conditions of hydrolysis.

The results were in full agreement with the expected sequence. Moreover, all the amino acids of the fully assembled peptide were indeed in the right configuration.

### Example 2

### Preparation of CBP501 Formulation Dosage Form: Injection, Powder, Lyophilized, for Solution (INJ PWD LYO F/SOL)

CBP501-Drug Product is a lyophilized powder for solution to be administered intravenously is composed of

>100 mg CBP501-Drug Substance

>acetic acid (qs pH=4.0)

to be reconstituted with 10 mL of 5% Dextrose Injection, USP before administration.

CBP501-Drug Product is packed in neutral white type I glass vial USP 27 <661>, closed with bromo-butyl vacuum stoppers USP 27 <381>, sealed with a blue flip cap.

CBP501 Drug Substance was dissolved in Water for Injection (WFI) acidified with acetic acid. This solution was filtered, filled in the vials and freeze-dried. The vials were automatically crimped, gathered in polypropylene boxes and transferred.

The label was completed for the lot number and the manufacturing date and affixed on each vial.

### Example 3

### Experiments-1

### Background

Most cancer cells are dependent on the G2 checkpoint to survive with DNA damage. The stable peptide CBP501 shows selective G2 checkpoint abrogation, with activity in various tumor models, alone and combined with DNA damaging agents. CBP501 was evaluated in 2 phase I studies, single agent and with cisplatin (CDDP), determining maximum tolerated dose (MTD), dose limiting toxicity (DLT), safety and pharmacokinetics (PK).

### Methods

CBP501-Drug Substance (acetate salt) was used as CBP501.

CBP501 was given over 1 hr, via central catheter, with prophylactic anti-allergics due to histamine release syndrome in animals. PK was examined in cycle (cy) 1. MTD is the level below that where 2 of 3-6 patients (pts) have DLT during cy 1-2. Study A: CBP501 day 1/8/15, q4w, initial dose 0.9 mg/m². Study B: q3w, initial CBP501/CDDP doses 3.6/50 mg/m².

### Results

Studies were run in 4 US centers from June-05 and still on going.

A) 30 pts treated, M/F: 16/14, median age 61 (25-82), performance status (PS) 0/1/2:9/20/1, median 4 prior lines, colon (5 pts), pancreas/biliary (6), ovarian (6), melanoma (2), renal (2), NSCLC (2), other (7). Median cy/pt: 2 (1-8). All pts discontinued due to progressive disease (PD). 1 DLT occurred (transient asymptomatic grade (Gr) 3 troponin elevation) at 22.5 mg/m² (top Dose Level (DL) given). No other Gr 3-4 adverse events (AEs) occurred; 13 pts (43%) had Gr 1-2 allergy. MTD was not reached. 2 pts (pancreas, ovarian) had stable disease (SD) for 7 cy.

B) 27 pts treated, M/F:14/13, median age 61 (31-81), PS 0/1:8/19, median 4 prior lines, mesothelioma (5), prostate (4), NSCLC (4), pancreas/biliary (3), esophageal (3), other (8). Two DLTs (Gr 3 allergic reaction) occurred at CBP501/CDDP 36.4/75 mg/m². No other Gr 3-4 AEs occurred. MTD was defined as 25 mg/m² CBP501, 75 mg/m² CDDP. Allergic reaction was the most common AE, observed in 12 (44%) pts. PR (unconfirmed) endometrial (7+cy, CA125 50% drop); SD, 2 mesothelioma (11+, 5), adenoid cystic (8), neuroendocrine (4+), ovarian (3+, CA125 50% drop), NSCLC (3+). PK: CBP501 Cmax and AUC showed dose-proportionality, and no difference between day 1 and 15 for both studies.

### Conclusion

CBP501 was well tolerated, as single agent and with CDDP. The main toxicity was dose-limiting allergic reaction. Promising signs of efficacy are seen in pts already exposed to platinum. Phase I-II studies of CBP501/pemetrexed/CDDP are planned.

Examples of the Arm include the following.

(Drug) pemetrexed+cisplatin+CBP501

### (i) CBP501

CBP501 for injection is provided in single dose vials (20 mg) containing a sterile lyophilized powder comprising CBP501 peptide acetate salt (peptide base units). For administration, vial contents are reconstituted in 5% Dextrose Injection, USP, and added to a 100 mL IV bag of 5% Dextrose Injection, USP.

### (ii) Pemetrexed

A commercial formulation of pemetrexed will be used, with reconstitution in 20 mL 0.9% sodium chloride solution for injection, then dilution to 100 mL.
(iii) Cisplatin
A commercial formulation will be used and will be diluted in 250 mL of normal saline for administration.

### (Method)

1. CBP501 25 mg/m² will be administered as an i.v. infusion of 1 hr.
2. Pemetrexed 500 mg/m² will be administered as an i.v. infusion over 10 min, immediately after the CBP501 infusion.
3. Cisplatin 75 mg/m² will be administered as a 1 hr i.v. infusion immediately after the pemetrexed infusion.

### (Eligibility)

Ages Eligible for Study: 18 Years and older

### Genders Eligible for Study: Both

### Accepts Healthy Volunteers: No

### (Inclusion Criteria)

(1) Signed informed consent obtained prior to initiation of any study-specific procedures
(2) Phase I: Histologically confirmed solid malignancy that is metastatic or unresectable and for which standard curative or palliative measures do not exist or are no longer effective or would otherwise be eligible for cisplatin and pemetrexed as first-line therapy
(3) Phase II: Histologically or cytologically confirmed diagnosis of malignant pleural mesothelioma (MPM), not amenable for radical resection, who has not received previous chemotherapy or other systemic treatment
(4) Measurable disease according to the modified Response Evaluation Criteria in Solid Tumors (RECIST, see below)
(5) Male or female patients aged at least 18 years

### (6) ECOG Performance Status (PS): 0-2

(7) Previous anticancer treatment must be discontinued at least 3 weeks prior to first dose of study treatment (6 weeks for mitomycin C; 6 weeks for anti-androgen therapy if discontinued prior to treatment initiation, with the exception of 8 weeks for bicalutamide)
(8) Life expectancy greater than 3 months with adequate organ function
(9) Female patients of child-bearing potential must have a negative pregnancy test and be using at least one form of contraception as approved by the Investigator for 4 weeks prior to the study and 4 months after the last dose of study drug. For the purposes of this study, child-bearing potential is defined as: “All female patients unless they are post-menopausal for at least one year or are surgically sterile”
(10) Male patients must use a form of barrier contraception approved by the investigator during the study and for 4 months after the last dose of study drug
(11) Ability to cooperate with the treatment and follow-up

### (Exclusion Criteria)

(1) Radiation therapy to more than 30% of the bone marrow prior to entry into the study
(2) Phase II only: Mesothelioma originating outside the pleura (e.g.: peritoneum)
(3) Absence of measurable lesions
(4) The patient has an ongoing or active infection, symptomatic congestive heart failure, unstable angina pectoris, symptomatic or poorly controlled cardiac arrhythmia, uncontrolled thrombotic or hemorrhagic disorder, or any other serious uncontrolled medical disorders in the opinion of the investigator.
(5) Any previous history of another malignancy within 5 years of study entry (other than cured basal cell carcinoma of the skin or cured in-situ carcinoma of the cervix)
(6) Presence of any significant central nervous system or psychiatric disorder(s) that would hamper the patient's compliance
(7) Evidence of peripheral neuropathy >grade 1 according to NCI-CTCAE Version 3
(8) Treatment with any other investigational agent, or participation in another clinical trial within 28 days prior to study entry
(9) Pregnant or breast-feeding patients or any patient with childbearing potential not using adequate contraception
(10) Known HIV, HBV, HCV infection
(11) Presence of CNS metastases

Furthermore, the effect of the present invention are explained in detail.

In this example, the primary objective was to establish the maximum tolerated dose (MTD) for the schedule being studied. Secondary objectives were:

(1) to establish the recommended doses (RD) for the same schedules,
(2) to evaluate the safety profiles,
(3) to evaluate the pharmacokinetic profiles of CBP501 and cisplatin,
(4) to evaluate patients for evidence of anti-tumor activity (single-agent study only), to determine phosphorylation levels of target proteins in tumor tissues, and assess the effect of CBP501 on biological surrogate markers of efficacy.

### Study Design

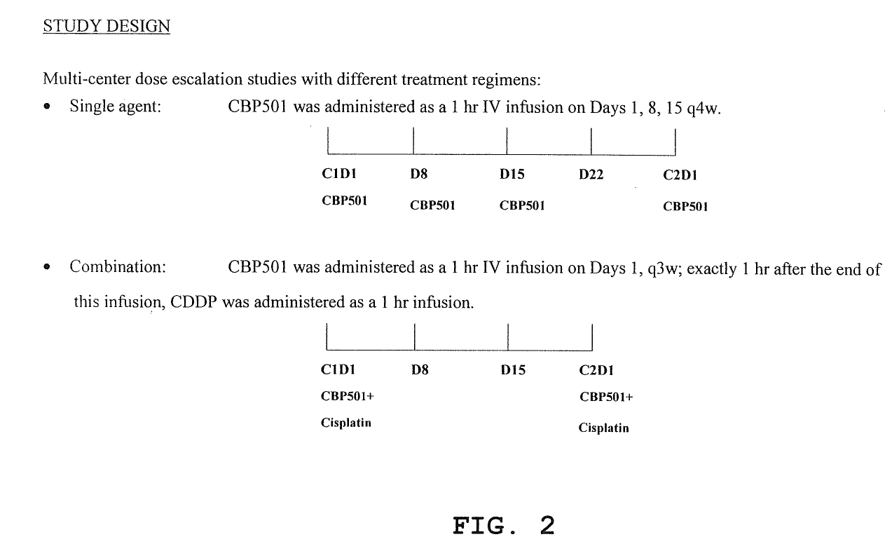

Multi-center dose escalation studies with different treatment regimens were shown in FIG. 2.

### Dose Escalation

In the combination study, cisplatin escalation was to be performed prior to CBP501 escalation, from a starting dose of 50 mg/m²; dose escalation to 75 mg/m² was to proceed in the absence of dose-limiting toxicity (DLT) in more than 1 out of 3-6 patients during the first 2 cycles.

In the single agent study and the second stage of the combination study, the dose of CBP501 was to be escalated by 100% if no toxicity was observed in the first 2 cycles of the previous dose level, by 50% in the event of Gr 1 toxicity, and by 33% in the event of Gr 2-4 toxicity. The initial dose of CBP501 was 0.9 mg/m² in the single agent and 3.7 mg/m² in the combination study.

A minimum of 3 patients were to be enrolled at each dose level, and 6 in the event of DLT. CBP501 escalation was to continue if fewer than 2 out of 3-6 patients in a dose level experienced DLT during the first 2 cycles.

### Anti-Allergy Prophylaxis

Following observation of anaphylactoid reactions in non-clinical studies and the first dose levels of both phase I studies, a prophylactic anti-allergy regimen was implemented in both studies, consisting of:

(1) Dexamethasone 4 mg orally, twice per day, the day before and the day after CBP501 infusion,
(2) Dexamethasone 8 mg IV immediately prior to CBP501 infusion,
(3) Diphenhydramine (DPH) 50 mg IV and Ranitidine 50 mg IV prior to CBP501 infusion, and
(4) Loratadine (10 mg) PO the day before, the day of CBP501 administration and the day after.

### Dose Limiting Toxicity (DLT) and Maximum Tolerated Dose (MTD)

Dose Limiting Toxicity is defined as any of the following events:

(1) Neutropenia Gr 4 lasting 7 days (5 days in combination study) or Gr 3-4 neutropenia with fever and/or infection,
(2) Thrombocytopenia Gr 4 (or grade 3 with bleeding),
(3) Treatment-related non-hematological toxicity Gr 3 or 4 (excluding Gr 3 vomiting or diarrhea with sub-optimal prophylactic and curative treatment), and
(4) Dosing delay greater than 2 weeks or treatment discontinuation due to treatment-emergent adverse events or related severe laboratory abnormalities.

The MTD was defined as the dose level below that in which DLT was observed during the first 2 cycles in at least 2 out of the 3-6 patients assessable for determination of MTD. The MTD was to be the RD for future studies. Patients evaluable for MTD were those who completed 2 treatment cycles or experienced DLT during the first 2 cycles.

In the combination study, which was run in parallel with the single-agent study, the dose of CBP501 overtook that in the singe-agent study, at which point the single agent study was terminated prior to the MTD being established.

### Cardiac Monitoring

Both studies implemented cardiac monitoring, including:

(1) Echocardiogram or MUGA scan (baseline and every 2 cycles),
(2) Cardiac enzymes (CPK-MB, CPK-MM, troponin; baseline and prior to each administration), and
(3) ECG (before infusion, end of infusion, and 1 hr after infusion end).

After 16 patients had been included, a central review of ECGs was implemented to ensure reliability of these parameters, primarily QTc.

### Pharmacokinetic Assessments

In the single agent study, PK was examined in cycle 1. Samples were collected on Days 1 and 15 before the start of the infusion, then at various time points over 24 hr from the start of the infusion, as described below.

In the combination study, samples for CBP501 pharmacokinetic analysis for both single-agent and combination treatment were taken before the start of the CBP501 infusion (time 0), 30 minutes after the CBP501 infusion start, at the end of the CBP501 infusion, then 30 minutes, 1, 2, 3, 5, 7 and 23 hours after the end of the CBP501 infusion on Day −7 and Day 1 of the first cycle. Blood samples for cisplatin analysis were collected before the start of the cisplatin infusion, at the end of the cisplatin infusion, and at 1, 3 and 21 hours after the end of the cisplatin infusion on Day 1 of the first cycle.

### Pharmacodynamic Assessments

Pharmacodynamic assessment was originally implemented with an ELISA method to detect Cdc25C and its phospho-Ser 216 in PBMC. During the study, it was decided that the signals detected were artifacts and sample collection was halted. Efforts to re-validate the method are ongoing.

### Eligibility Criteria

Standard inclusion criteria were used for both studies, including the following:

(1) Pathologically-confirmed, locally advanced or metastatic solid tumors, refractory to standard therapy or for which conventional therapy is not reliably effective,
(2) Presence of measurable disease according to RECIST criteria was not required, but was recommended in additional patients entered at MTD,
(3) Age ≧18 years; Eastern Cooperative Oncology Group (ECOG) PS 0-2; Life expectancy >3 months,
(4) Adequate organ function (hematological, hepatic, renal, metabolic), and
(5) Signed informed consent

Standard exclusion criteria were used for both studies, including the following:

(1) Prior chemotherapy with nitrosoureas or high dose is carboplatin (AUC >6), prior mitomycin C cumulative dose ≧25 mg/m², prior bone marrow transplant or intensive chemotherapy with stem cell support, and
(2) Patients with active CNS metastasis.

In the combination study, criteria were modified as follows:

ECOG PS 0-1; the interval between previous anticancer treatment and study entry was reduced from 4 weeks to 3 weeks; minimum creatinine clearance was raised from ≧60 to ≧70 mL/min; patients with evidence of peripheral neuropathy Gr >1 were excluded.

### Patient Characteristics

Accrual is completed in both studies.

CBP501 Single Agent (Single Agent): 30 patients were included and treated in 8 dose levels, with the highest dose level being 22.5 mg/m². A total of 68 cycles were administered, with a median of 2 cycles per patient.
CBP501/Cisplatin (combination): 33 patients were included and treated in 7 dose levels, with the highest dose level being 36.4 mg/m² CBP501 and 75 mg/m² cisplatin. To date, a total of 114 cycles have been administered, with a median of 2 cycles per patient. Seven patients are still on-study and the RD is being confirmed.

In both studies, patients were in good physical condition, with just 1 patient having ECOG PS of 2 at baseline.

TABLE 6

| | Single-agent | Combination |
|---|---|---|
| Patients treated | 30 | 33 |
| Median age (range) | 62 (25-82) | 61 (31-80) |
| ECOG PS 0/1/2 | 9/20/1 | 11/22/0 |

| | | | | |
|---|---|---|---|---|
| Tumor types: | Ovarian | 6 | Ovarian | 3 |
| | Pancreas/ | 6 | Mesothelioma | 7 |
| | biliary | | NSCLC | 5 |
| | Colorectal | 5 | Prostate | 4 |
| | Other | 13 | Pancreas | 3 |
| | | | Oesophageal/ | 3 |
| | | | gastric | |
| | | | Other | 8 |

| | | |
|---|---|---|
| Median no. of | 2 | 2 |
| organs involved | | |
| Median prior lines | 4 (1-11) | 4 (0-7) |
| of therapy (range) | | |
| Median no. of | 2 (1-9) | 4 (1-13) |
| cycles study | | |
| treatment (range) | | |

In the single agent study, the majority of patients (26 pts, 87%) discontinued from the study due to progressive disease; 2 patients discontinued following an investigator's decision, 1 patient withdrew consent, and one patient died due to an AE (sepsis, considered unrelated to treatment). In the is combination study, 17 patients (52%) discontinued due to disease progression. Nine patients discontinued due to treatment-related AEs: 3 patients with allergic reaction, 3 patients with peripheral neuropathy/ototoxicity, and 3 patients with persistent nausea/vomiting. Seven patients are currently still on treatment.

### Safety

In both studies, dose increments were determined by the incidence of Gr 1-2 allergic reactions.

### MTD-DLTs

### Single Agent Study

The highest dose level explored was 22.5 mg/m². At this dose level, one patient experiences one DLT: transient Gr 3 troponin increase during the treatment cycle. The MTD was not reached in this study as dose escalation was halted when the dose was overtaken by the combination study.

### Combination Study

The highest dose level explored was 36.4 mg/m² CBP501 and 75 mg/m² cisplatin. At this DL two DLTs were reported in 2 patients: both patients experienced Gr 3 allergic reaction (acute infusion reaction). The MTD in this study was thus defined as 25 mg/m² CBP501 and 75 mg/m² cisplatin.

TABLE 7

| | | | | Combination | | | |
|---|---|---|---|---|---|---|---|
| Single-agent | | | | (CBP501/cisplatin) | | | |
| Dose* | Pts | Cycles | DLT | Dose | Pts | Cycles | DLT |
| 0.9 | 4 | 7 | — | 3.6/50 | 3 | 6 | — |
| 1.8 | 3 | 5 | — | 3.6/75 | 3 | 8 | — |
| 3.6 | 4 | 14 | — | 7.2/75 | 3 | 24 | — |
| 7.2 | 3 | 6 | — | 10.8/75 | 5 | 14 | — |
| 9.5 | 3 | 7 | — | 16.2/75 | 4 | 10 | — |
| 12.7 | 3 | 11 | — | 25/75 | 9 | 26 | — |
| 16.9 | 3 | 7 | — | 36.4/75 | 6 | 24 | 2 |
| 22.5 | 7 | 11 | 1 | — | — | — | — |

*dose in mg/m²

The principle AEs reported in the study are summarised in the Table 8. The most frequently reported AE was the allergic reaction related to =501 infusion. In both studies, Gr 3-4 events were rare.

TABLE 8

| | Single-agent (n = 30) | | | Combination (n = 33) | | |
|---|---|---|---|---|---|---|
| | Gr 1-2 | Gr 3 | Gr 4 | Gr 1-2 | Gr 3 | Gr 4 |
| Hematological | | | | | | |
| Neutropenia | 3 | — | — | 7 | 1 | — |
| Thrombocytopenia | 4 | — | — | 3 | — | — |
| Anemia | 22 (16*) | — | — | 21 (14*) | 1 (1*) | 1 |
| Non-hematological | | | | | | |
| Allergy** | 18 | — | — | 14 | 2 | — |
| Nausea/Vomiting | 5 | — | — | 16 | 1 | — |
| QTc prolongation | 10 | — | — | 15 | 1 | — |
| Fatigue | 14 | 1 | — | 12 | 3 | — |
| Mucositis | — | — | — | 3 | — | — |
| Peripheral neuropathy | — | — | — | 5 | — | — |
| Increased troponin | — | 1 | — | — | — | — |
| Abdominal pain | 7 | 2 | — | 2 | — | — |
| Diarrhea | 5 | 1 | — | — | — | — |
| Headache | 3 | — | — | — | — | — |

*Number of patients with baseline anemia
**Includes infusion reaction, flushing, rash, etc.

The principle AE observed during both of these studies was allergic reaction, characterized by rash, hot flushes, and urticaria, starting in the head, neck and upper chest 10-60 minutes into the infusion; no respiratory or hemodynamic changes were observed. Incidence and severity of allergic reaction showed some dose-dependency. Use of the prophylaxis regimen attenuated the reaction but did not prevent it.

TABLE 9
Patients with allergic reaction

| Single | | | | | Combination (CBP501/cisplatin) | | | | |
|---|---|---|---|---|---|---|---|---|---|
| CBP501* | Pts | Gr1 | Gr2 | Gr3 | CBP501* | Pts | Gr1 | Gr2 | Gr3 |
| 0.9 | 4 | 1 | — | — | 3.6/50 | 3 | — | — | — |
| 1.8 | 3 | 1 | — | — | 3.6/75 | 3 | — | — | — |
| 3.6 | 4 | — | 1 | — | 7.2/75 | 3 | — | 2 | — |
| 7.2 | 3 | — | 3 | — | 10.8/75 | 5 | 3 | 1 | — |
| 9.5 | 3 | 1 | 2 | — | 16.2/75 | 4 | — | 3 | — |
| 12.7 | 3 | — | — | — | 25/75 | 4 | — | 2 | — |
| 16.9 | 3 | 1 | 1 | — | 36.4/75 | 6 | — | 3 | 2 |
| 22.5 | 7 | 3 | 4 | — | — | — | — | — | — |
| TOTAL | 30 | 7 | 11 | — | TOTAL | 28 | 3 | 11 | 2 |

*dose in mg/m²

### Cardiac Monitoring

One patient with increased troponin

QTcB findings: 1 pt with QTcB >500 msec
2 pts with prolongation >60 msec
   QTcB: Bazett's corrected QT interval

Cardiac monitoring was performed in all treated patients, in both studies.

Cardiac enzymes were unchanged from baseline in all patients except one. One patient treated at DL 8 in the is single-agent study experienced asymptomatic Gr 3 troponin elevation during the first cycle. This patient was a 66-year-old female with no cardiac history and no prior anthracycline treatment. No concurrent LVEF or ECG changes were observed. Troponin returned to normal levels within one week. This event was reported as a DLT and an SAE.

Centralized ECG review was performed for 16 patients in the single-agent study and 24 patients in the combination study. QTc prolongation by >60 msec was observed in 4 patients. In the single agent arm, one patient with significant cardiac antecedents had an increase >60 msec after the second infusion of the first cycle. In the combination arm, 3 patients had QTc prolongations >60 msec: amongst these, two patients had significant cardiac precedents, while the third had hypertension, hypomagnesemia and hypothyroidism. In two patients, these QTc prolongations occurred during the third treatment cycle, while in the third, prolongation >60 msec (QTc >500 msec) was observed during the 9th and 13th cycles.

TABLE 10

| | QTc > 60 ms above baseline | QTc > 500 ms |
|---|---|---|
| Single agent (16 pts) | 1 | 0 |
| Combination (24 pts) | 3 | 1 |

### Antitumor Activity

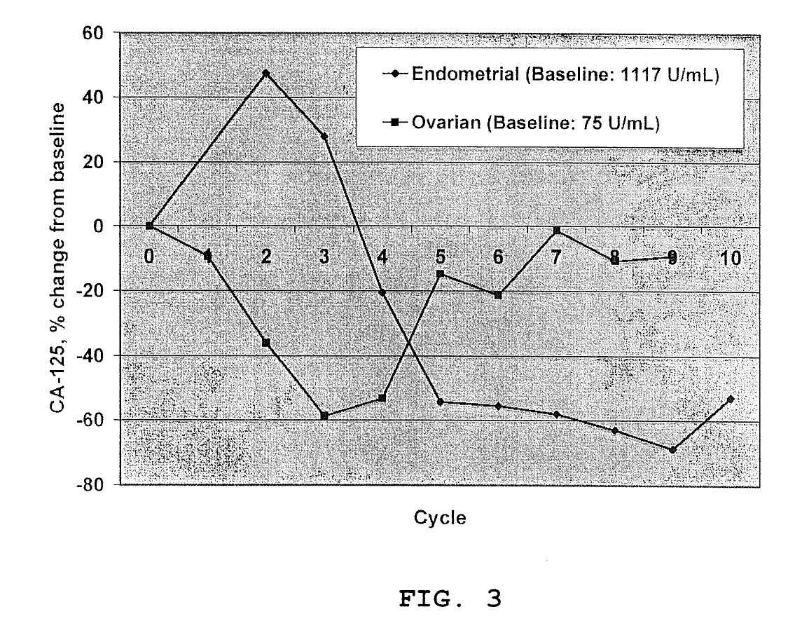

Promising signs of antitumor activity were observed, particularly in the combination study. Details of these patients are provided below (see, also FIG. 3).

TABLE 11

| | | | | | | |
|---|---|---|---|---|---|---|
| CBP501 | | | Prior | | Response | Reason for |
| dose | Tumor | Sites* | lines | Cycles | (RECIST) | D/C** |

Single agent

| | | | | |
|---|---|---|---|---|
| 3.6 | Ovarian | 8 | SD | PD |
| 12.7 | Pancreas | 7 | SD | Death |
| | | | | (sepsis) |

Combination

| | | | | | | |
|---|---|---|---|---|---|---|
| 7.2 | Pleural | Lu, LN | 2 | 13 | SD | Withdrew |
| | mesothelioma | | | | | consent |
| 7.2 | Salivary gland | Liv, | | 8 | SD | Toxicity |
| | | Lu, | | | | |
| | | Bone | | | | |
| 16.2 | Pleural | Liv, | 2 | 5 | SD | PD |
| | mesothelioma | Lu | | | | |
| 24.3 | Endometrial | Perit, | 3 | 11 | PR (c) | — |
| | carcinoma | Pelvis | | | | |
| 36.4 | Ovarian | | | 7⁺ | SD | — |
| | adenocarcinoma | | | | | |
| 36.4 | Neuroendocrine | Liv | 1 | 6 | SD | Withdrew |
| | | | | | | consent |
| 36.4 | NSCLC | Lu, LN | 5 | 3 | SD | Withdrew |
| | | | | | | consent |

*Lu: lungs; LN: lymph nodes; Liv: liver; Perit: peritoneum
**Discontinuation
+still on treatment

### Pharmacokinetics

TABLE 12
Single Agent (mean and CV)

| | Dose | | N | Cₘₐₓ | | T_{1/2} | | AUC_{0-inf} | | Cl | | Cl | |
|---|---|---|---|---|---|---|---|---|---|---|---|---|---|
| DL | (mg/m²) | Day | Pts | (ng/mL) | | (h) | | (ng · h/mL) | | (mL/h/kg) | | (mL/h/m²) | |
| I | 0.9 | 1 | 4 | 121 | 7 | 0.7 | 56 | 178 | 21 | 123 | 30 | 5240 | 20 |
| I | 0.9 | 15 | 3 | 138 | 14 | 0.6 | 29 | 188 | 19 | 108 | 12 | 4908 | 18 |
| II | 1.8 | 1 | 3 | 516 | 37 | 1.2 | 34 | 909 | 35 | 55 | 26 | 2197 | 43 |
| II | 1.8 | 15 | 3 | 482 | 43 | 1.6 | 94 | 866 | 54 | 62 | 36 | 2506 | 50 |
| III | 3.6 | 1 | 4 | 1062 | 26 | 1.7 | 35 | 1782 | 30 | 52 | 16 | 2908 | 71 |
| III | 3.6 | 15 | 3 | 1068 | 36 | 1.6 | 7 | 1881 | 45 | 50 | 28 | 2176 | 41 |
| IV | 7.2 | 1 | 3 | 1916 | 51 | 1.3 | 48 | 4392 | 38 | 49 | 27 | 1802 | 37 |
| IV | 7.2 | 15 | 3 | 2008 | 19 | 1.1 | 14 | 3664 | 23 | 56 | 12 | 2041 | 25 |
| V | 9.57 | 1 | 3 | 2890 | 27 | 2.5 | 87 | 6480 | 37 | 38 | 39 | 1599 | 31 |
| V | 9.57 | 15 | 2 | 2413 | 30 | 1.2 | 0 | 5139 | 31 | 47 | 41 | 1954 | 31 |
| VI | 12.73 | 1 | 3 | 2731 | 19 | 1.4 | 11 | 6340 | 21 | 59 | 16 | 2081 | 24 |
| VI | 12.73 | 15 | 3 | 3083 | 4 | 2.4 | 73 | 7661 | 12 | 48 | 2 | 1677 | 12 |
| VII | 16.93 | 1 | 2 | 4924 | 31 | 2.0 | 35 | 14257 | 55 | 37 | 53 | 1395 | 55 |
| VII | 16.93 | 15 | 3 | 4637 | 18 | 2.9 | 51 | 12595 | 18 | 36 | 15 | 1372 | 17 |
| VIII | 22.5 | 1 | 7 | 6077 | 20 | 3.2 | 43 | 16509 | 28 | 40 | 31 | 1458 | 28 |
| VIII | 22.5 | 15 | 5 | 7492 | 25 | 4.0 | 12 | 20225 | 31 | 35 | 41 | 1206 | 32 |

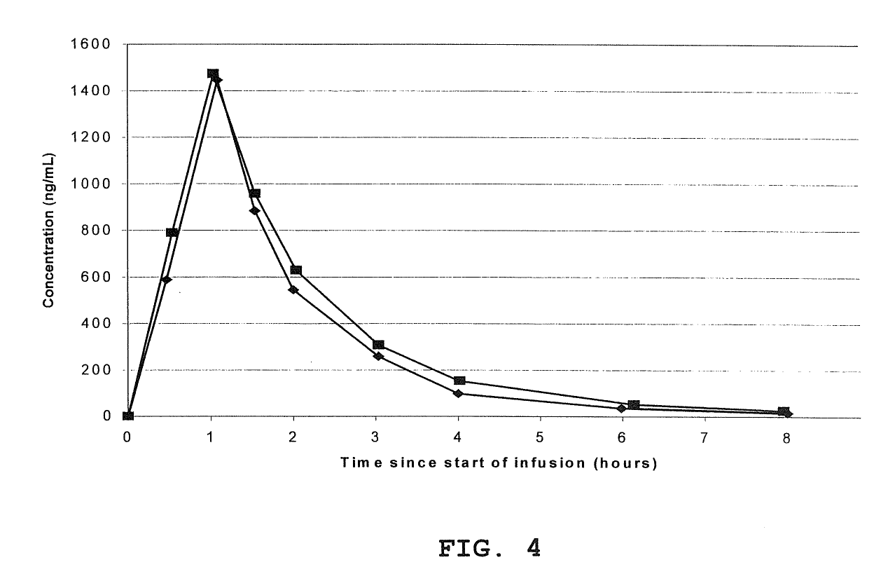

Characteristic CBP501 concentration-time profile since the start of CBP501 infusion was depicted (FIG. 4).

TABLE 13
Combination: CBP501 (mean and CV)

| | Dose | | N | Cₘₐₓ | | T_{1/2} | | AUC_{0-inf} | | Cl | | Cl | |
|---|---|---|---|---|---|---|---|---|---|---|---|---|---|
| DL | (mg/m²) | Day | Pts | (ng/mL) | | (h) | | (ng · h/mL) | | (mL/h/kg) | | (mL/h/m²) | |
| I | 3.6 | 1 | 6 | 734 | 18 | 3.9 | 162 | 1625 | 51 | 63 | 35 | 2526 | 29 |
| II | 7.2 | 1 | 3 | 1772 | 28 | 1.7 | 4 | 2821 | 32 | 75 | 45 | 2752 | 35 |
| m | 10.8 | 1 | 5 | 2546 | 23 | 1.7 | 59 | 5351 | 39 | 53 | 34 | 2198 | 26 |
| IV | 16.2 | 1 | 3 | 3490 | 29 | 2.8 | 92 | 7636 | 4 | 54 | 10 | 2124 | 4 |
| V | 24.3 | 1 | 2 | 6830 | 17 | 4.1 | 5 | 20222 | 16 | 34 | 15 | 1217 | 16 |
| VI | 36.45 | 1 | 5 | 9492 | 18 | 3.7 | 19 | 39415 | 25 | 25 | 25 | 971 | 24 |

TABLE 14
Combination: cisplatin (mean and CV)

| | Dose | N | Cₘₐₓ | | T_{1/2} | | AUC_{0-inf} | | Cl | |
|---|---|---|---|---|---|---|---|---|---|---|
| | (mg/m²) | Pts | (ng/mL) | | (h) | | (ng · h/mL) | | (L/h) | |
| Total Platinum | 50 | 3 | 2617 | 5 | 30.0 | 29 | 75455 | 37 | 1.5 | 41 |
| | 75 | 21 | 3662 | 19 | 39.2 | 52 | 136821 | 48 | 1.2 | 42 |
| Ultrafiltrate | 105 | 3 | 1363 | 21 | 6 | 5 | 3791 | 6 | 28 | 5 |
| Platinum | 126 | 21 | 2268 | 37 | 8 | 65 | 5259 | 27 | 28 | 29 |

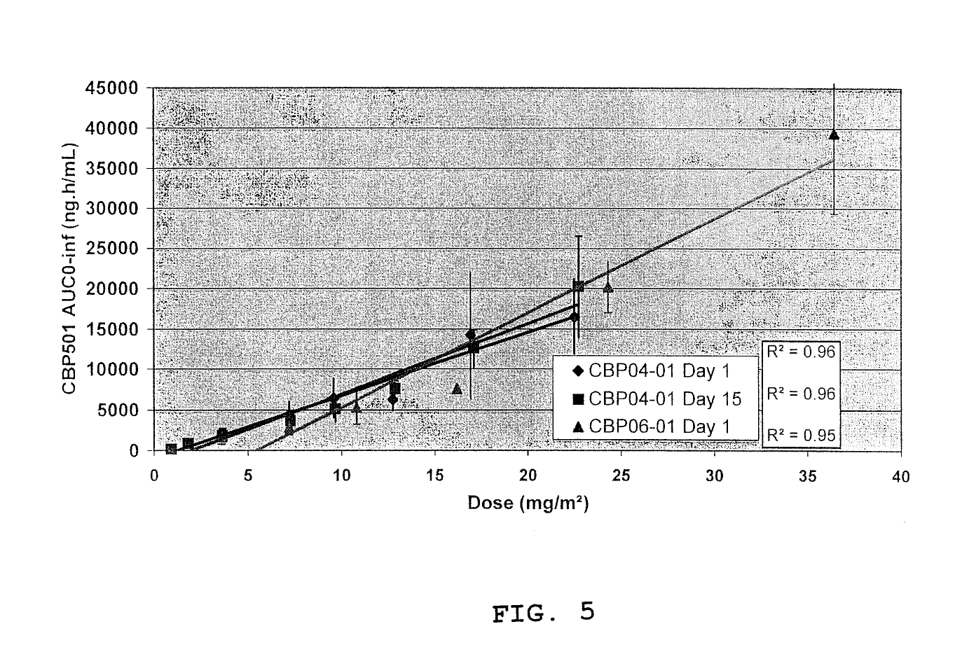

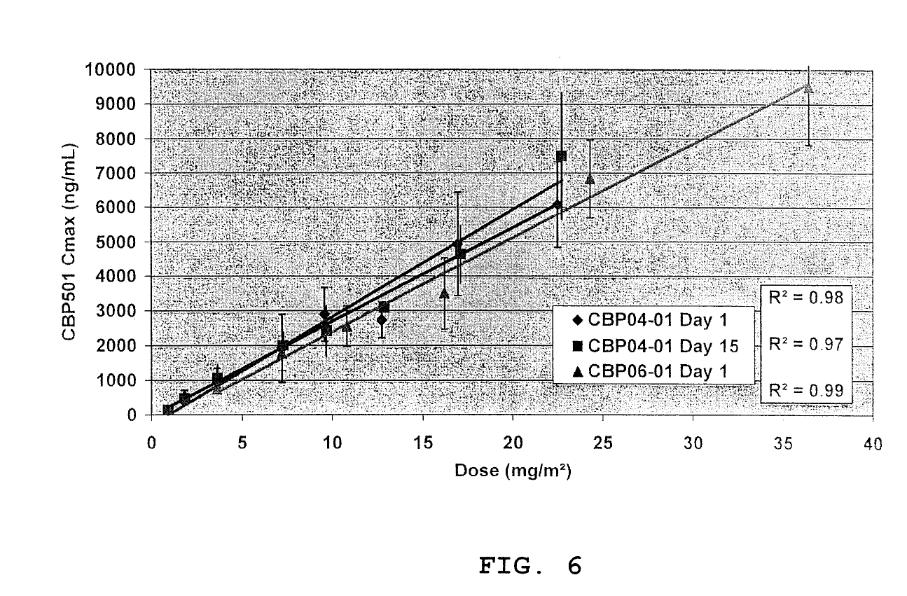

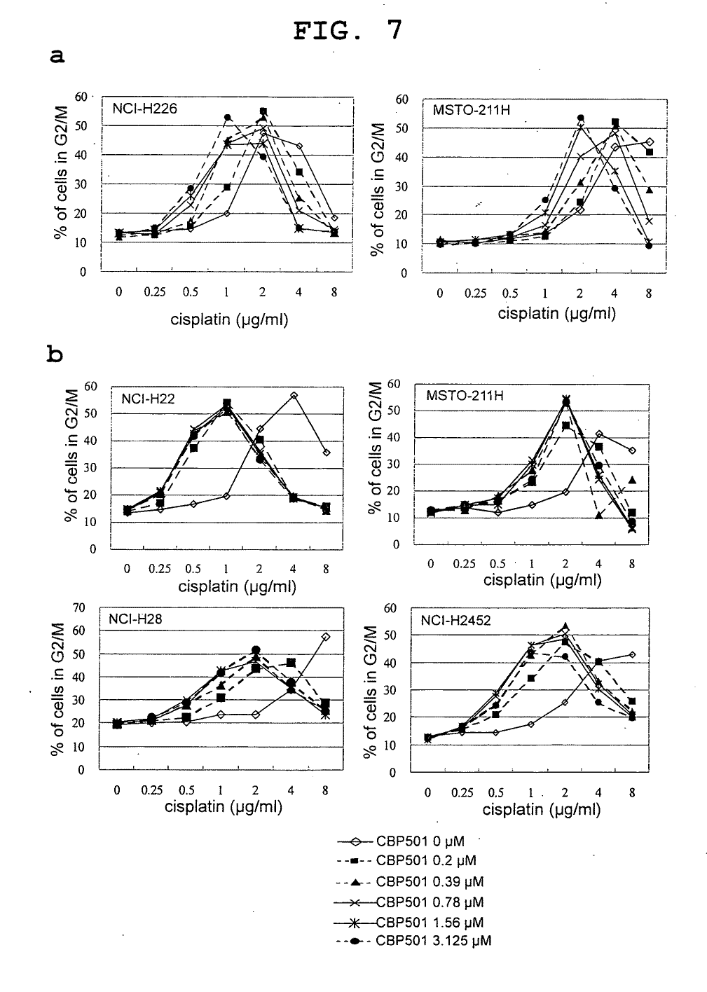

Cₘₐₓ and AUC_{0-∞} were observed to show dose-proportionality over the dose range evaluated on days 1 and 15 for single agent administration (CBP04-01) and in combination with cisplatin (CBP06-01) (see FIGS. 5 and 6).

Inter-patient variability within dose levels was moderate, with a coefficient of variation generally in the range 20%-40%. The observed tendency for a longer CBP501 half-life with increasing dose may be attributable to the measurability of later time points at higher dose levels. Clearance was observed to decrease with dose (R²=0.73 for linear interpolation in the combination study), despite dose-proportionality for AUC_{0-∞}.

No consistent differences were observed between PK on day 1 and day 15 or between single-agent and combination administration.

### Conclusions-1

The MTD in the single-agent study was not reached; the MTD in the combination study was 25 mg/m² CBP501 and 75 mg/m² cisplatin—this is the recommended dose for further clinical trials.

CBP501 was well tolerated, as single agent and combined with CDDP. The principle toxicity in both studies was allergic reaction. This was alleviated but not eradicated by the prophylactic regimen. Treatment-related SAEs were rare.

Promising signs of activity were observed, particularly in the combination study, notably in patients previously exposed to platinum. A partial response was reported in a patient with endometrial adenocarcinoma (CBP501/CDDP); stable disease lasting >4 months was reported in 5 patients treated in the combination study.

Dose-proportionally of Cmax an AUC_{0-inf} was observed over 40-fold dose range.

A new phase I-II study with CBP501 in combination with cisplatin/pemetrexed in mesothelioma patients is ongoing.

### Example 4

### Experiments-2

### Materials and Methods

Cell culture and reagents

Cells were cultured in various media, including RPMI1640 (Sigma-Aldrich, St. Louis, Mo.) for MSTO-211H, RPMI1640 supplemented with 4.5 g/L D-glucose (Sigma-Aldrich), 10 mM HEPES (Sigma-Aldrich) and 1 mM sodium pyruvate (Sigma-Aldrich) for NCI-H226, NCI-H28, and NCI-H2452, DMEM (Sigma-Aldrich) with 2.5% horse serum (Invitrogen) for MIAPaCa2, McCoy's 5A (Invitrogen) for HT29, and EBM-2 with hydrocortisone, VEGF, ascorbic acid, gentamicin, amphotericin B, hFGF-B, R3-IGF-1, heparin and hEGF (Sanko Junyaku, Tokyo, Japan) for HUVEC cells. Media was supplemented with 10% fetal bovine serum (Invitrogen Co., Carlsbad, Calif.) and cells were cultured at 37° C. with 5% CO₂/air. CBP501 was manufactured by Lonza Braine SA (Braine-l'Alleud, Belgium). Bleomycin (BLM) and cisplatin (CDDP) were purchased from Wako (Osaka, Japan) and Sigma-Aldrich, respectively.

### Cell Cycle Analysis

Cells were plated in 24-well plates and incubated for 24 hr, and were subsequently treated with or without bleomycin or cisplatin in the presence or absence of CBP501 at the indicated concentration for the indicated duration. The cells were harvested and stained with Krishan's solution (0.1% sodium citrate, 50 μg/ml propidium iodide, 20 μg/ml RNase A, 0.5% NP-40), and then analyzed by FACSCalibur (Becton Dickinson, N.J., U.S.A.) with the CELLQuest program (Becton Dickinson).

### Antibodies

The following antibodies were purchased from the respective companies: anti-γ-H2AX (Millipore, Billerica, Mass.), anti-ATM p-Ser1981 and anti-DNA-PKcs p-Thr2609 (Rockland, Gilbertsville, Pa.), anti-NBS1 p-Ser343 and anti-SMC1 p-Ser957 (Abcam, Cambridge, Mass.), anti-CHK1 p-Ser317, anti-CHK2 p-Thr68, anti-MAPKAPK2 p-Thr222, anti-CDC25C p-Ser216, anti-Histone H2AX and anti-β-Actin (Cell Signaling Technology, Danvers, Mass.), anti-CHK1 (Santa Cruz, Santa Cruz, Calif.), anti-CHK2 (Epitomics, is Burlingame, Calif.), and anti-CDC25C (BD Biosciences, San Jose, Calif.). Anti-γ-H2AX and anti-ATM p-Ser1981 were purchased from Cell Signaling technology and used in western blot analysis.

### Intracellular Platinum Concentration

NCI-H226 cells were treated with CBP501 (0.2, 2.0 μM) and cisplatin (1, 3, 9 μg/ml) for 3 hr, and harvested, or replenished with fresh media, incubated for 45 hr and then harvested. HUVEC, HT29, and MIAPaCa2 cells were treated with 3 μg/ml of cisplatin with or without 10 μM of CBP501 for 3 hr, harvested, and genomic DNA was extracted with phenol, chloroform and ethanol.

The cells or extracted DNA were dissolved by heating after adding nitric acid and hydrogen peroxide, and the samples were then diluted with aqua regalis of 4 vol. Platinum concentrations were measured by ICP-MS (Inductively Coupled Plasma Mass Spectrometer) analysis at Toray Research Center, Inc. (Shiga, Japan).

### Confocal Immunofluorescence Analysis

Cells cultured in 96-well plates were treated with bleomycin (0.4 μg/ml) and CBP501 (0.2 or 2 μM) for the indicated time. The cells were fixed in ice-cold methanol at −20° C. for 20 min, and blocked with PBS containing 5% BSA for 45 min. Cells were then incubated with anti-γ-H2AX, anti-ATM p-Ser1981, anti-DNA-PKcs p-Thr2609, anti-NBS1 p-Ser343, or anti-SMC1 p-Ser957 antibodies overnight at 4° C., followed by incubation with Alexa 594-conjugated secondary antibody (Invitrogen, Carlsbad, Calif.) for 1 hr at room temperature in the dark. Cell nuclei were stained using Hoechst 33342. Fluorescence was visualized and analyzed using Becton Dickinson Pathway450. The intensity of the nuclei and number of foci was assessed by ATTOVision and IPLab software (Becton Dickinson), respectively.

### Western Blot Analysis

Cells (at 50% confluence) were treated with or without bleomycin or cisplatin in the absence or presence of CBP501 at the indicated concentration for the indicated time. The cells were harvested and lysed in a lysis buffer [50 mM Tris-HCl (pH 8.0), 5 mM EDTA (pH 8.0), 100 mM NaCl, 0.5% NP-40, 2 mM DTT, 50 mM NaF, 1 mM Na₃VO₄, 1 μM microcystin, proteinase inhibitors cocktail (Roche, Mannheim, Germany)] for 30 min on ice and the lysates were clarified by centrifugation at 15,000 rpm for 20 min at 4° C. Protein concentration of the supernatants was evaluated using a detergent-compatible protein assay kit (Bio-Rad, Hercules, Calif.) according to the manufacturer's instructions. Whole cell lysates (60 μg) were run on a 10-12% SDS-PAGE gel, and transferred onto polyvinylidene difluoride (PVDF) membranes (Bio-Rad). The membrane was blocked at room temperature for 1 hr in TBST (0.1% Tween 20 in TBS) containing 2% ECL Advance Blocking Reagent (GE healthcare, Waukesha, Wis.) and incubated with primary antibody overnight at 4° C. The membrane was incubated with anti-peroxidase conjugated secondary antibody (Cell Signaling) for 1 hr at room temperature and analyzed using the enhanced chemiluminescence detection system (ECL Advance Western Blotting Detection Kit, GE healthcare).

### Water Soluble Tetrazolium (WST) Analysis

The cells were seeded at 5×10³ cells/well in 96-well plates and treated with cisplatin with or without the indicated dose of CBP501. The medium was replaced 24 hr after treatment, the cells were incubated for an additional 48 hr, and WST-8 solution (Kishida Chemical Co., Osaka, Japan) was added to each well. After 4 hr of incubation at 37° C., the absorbance at 450 nm was measured by microplate reader (Molecular Devices, Calif.).

### Xenograft Model

A suspension of NCI-H226 cells was injected subcutaneously into the flanks of six weeks old male severe is combined immune deficiency (SCID) mice (Charles River Lab., Wilmington, Mass.). The size of tumors was measured three times a week using a caliper. The volumes were calculated using the following formula: volume (cm³)=[width² (mm)×length (mm)]/2000. The relative tumor volume was expressed as the Vₜ/V₀ index, where Vₜ is the tumor volume on a measured day, and V₀ is the volume of the same tumor just before the first treatment. Mean relative tumor sizes with standard error of mean, SEM, were plotted. Body weight was measured three times weekly from the first treatment, and the weight change was expressed as a percentage of the initial weight. Animals were housed in accordance with guidelines from the Association for the Assessment and Accreditation of Laboratory Animal Care International, and the protocols were approved by the institutional animal care committee of CanBas Co., Ltd.

### Statistical Analysis

The statistical significance of the differences of the relative tumor volume between groups was determined by Student's t tests.

### Radio-Labeled CBP501

An N-terminal amino acid, p-benzoylphenylalanyl, was ³H labeled by Daiichi Kagaku Yakuhin Co., Ltd. (Tokyo, Japan).

### Results

### Experimental Example 1

### CBP501 Enhances Cisplatin-Induced G2/M Arrest in Cancer Cell Lines

Many cancer cells arrest at the G2/M phase of the cell cycle in response to DNA damage. A dose response curve between the amount of DNA damaging agent and the ratio of the cells in G2/M phase can be drawn, in which the G2/M population increases in proportion to the level of the DNA damaging agent. At a certain dose, the G2/M population begins to decrease once the DNA damage has reached a level to initiate apoptosis. FIG. 7 shows dose response curves of malignant pleural mesothelioma cell lines accumulating in G2/M phase in response to cisplatin, with or without CBP501 treatment. NCI-H226 and MSTO-211H cells were treated with CBP501 for 3 hr on day 1, cisplatin for 3 hr on day 2 and analyzed by FACS on day 4. The dose response curves of cells treated with both CBP501 and cisplatin shifted to the left compared to the curves of cells treated with cisplatin alone. (FIG. 7a).

Next, NCI-H226, MSTO-211H, NCI-H28 and HCI-H2452 cells were simultaneously treated with cisplatin and CBP501 for 3 hr and analyzed by FACS on day 3 (FIG. 7b). The result indicates that the dose level of CBP501 at 0.2 μl was already at the plateau level and the dose response curves shifted to the left by two to four folds in all four cell lines. Together these results suggest that CBP501 increased DNA damage, decreased DNA repair, or increased checkpoint signals to arrest in G2/M phase.

Similar results were obtained with bleomycin treatment but not with other DNA damaging anti-cancer medicines, such as doxorubicin, camptothecin or X-irradiation (data not shown). As previously reported in other cell lines (non-patent literature 14), CBP501 alone (up to 25 μM) did not alter the cell cycle distribution of these cells (data not shown).

### Experimental Example 2

### CBP501 Increased Intracellular Platinum Concentrations as Well as Platinum-DNA Adducts in the Cancer Cells

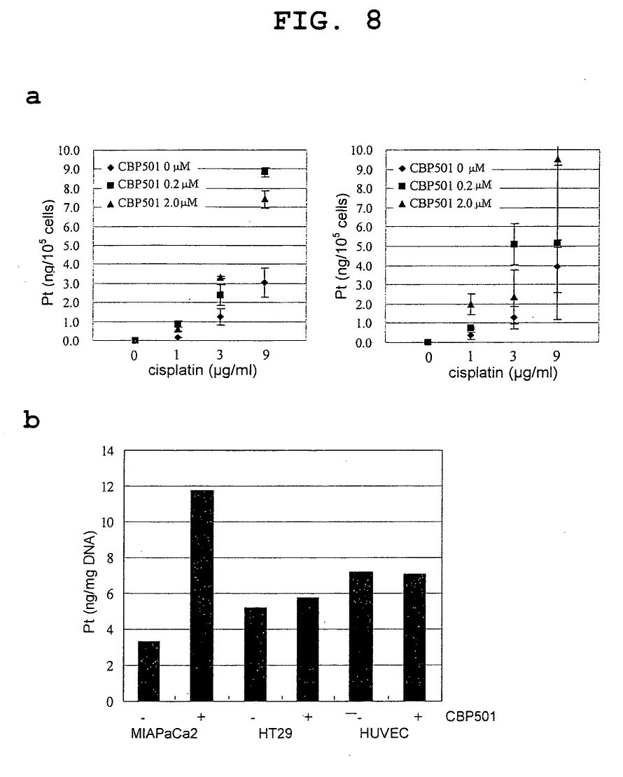

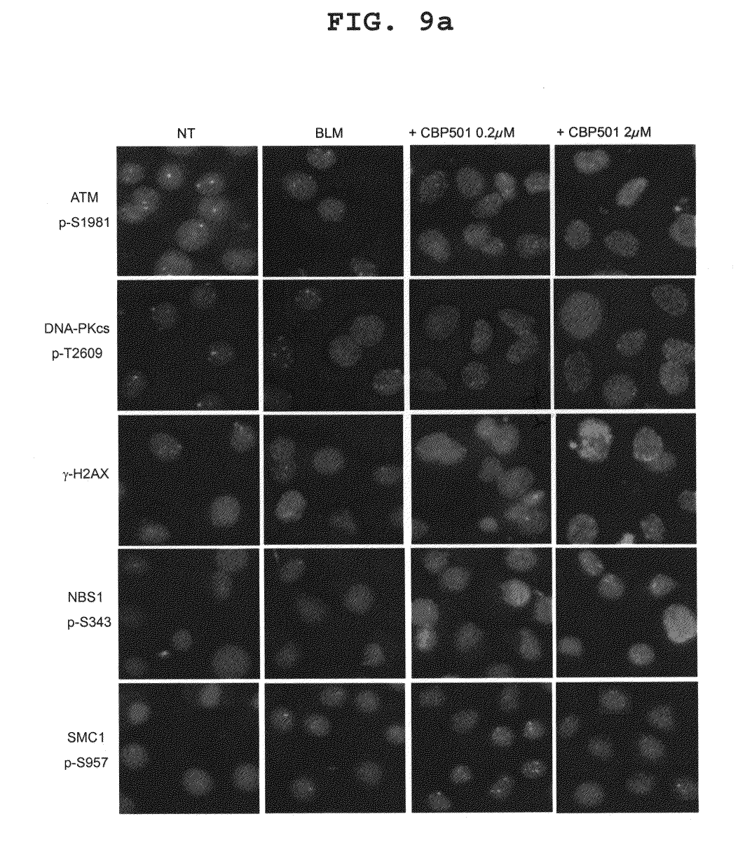

To examine the effect of CBP501 on the accumulation of cisplatin in cells, we investigated intracellular platinum concentrations. FIG. 8a shows the dose-dependent accumulation of platinum in NCI-H226 cells treated with cisplatin alone compared to co-treatment with CBP501. CBP501 increased the concentration of platinum in cells harvested after 3 hr of treatment (left panel) as well as additional 45 hr after the medium was changed at the 3 hr time point (right panel). The difference in concentrations between CBP501 minus and plus treatments did not significantly differ between the experiments, suggesting that the platinum concentration in the cells is more dependent on influx rather than efflux. We performed a similar study with MIAPaCa2, a pancreatic cancer cell line, HT29, a colon cancer cell line, and HUVEC cells, a human umbilical endothelial cell. Co-treatment of CBP501 with cisplatin increased the platinum concentration in MIAPaCa2 cells compared to cisplatin alone, but no effect was seen in HT-29 and HUVEC cells (data not shown). DNA-platinum adducts also increased in MIAPaCa2 cells but not in HT29 and HUVEC cells (FIG. 8b). The ratios of the amount of platinum on genomic DNA and whole cells with and without CBP501 treatment were roughly the same (data not shown).]

### Experimental Example 3

### CBP501 Enhanced the Damage Foci Formation by Bleomycin

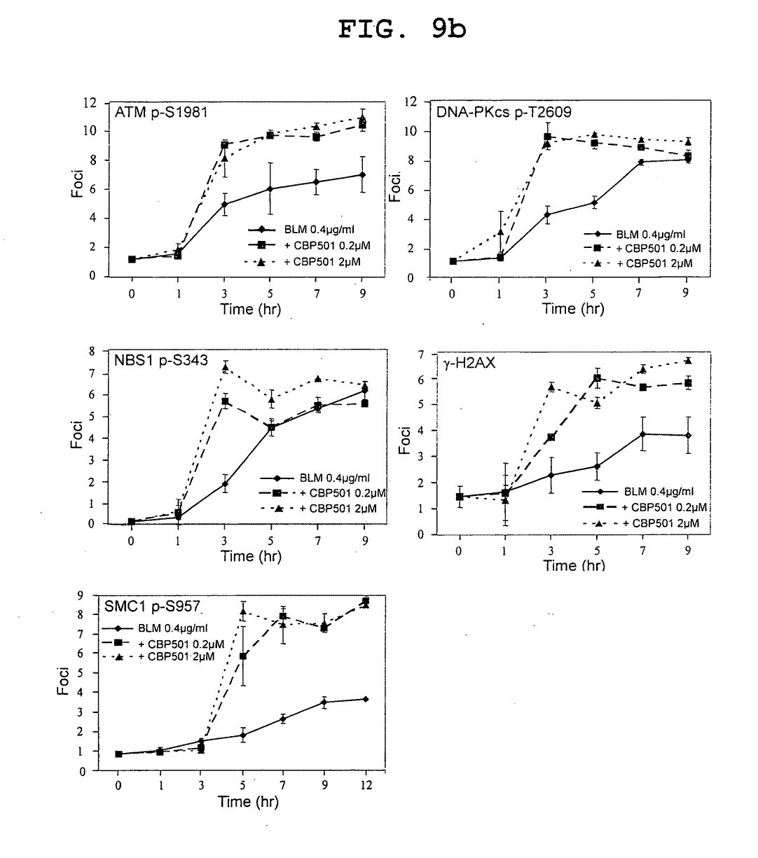

Bleomycin treatment causes DNA double strand breaks in cells, which are recognized by multiple DNA damage sensor proteins and complexes that form microscopically visible foci. Thus, next the present inventor analyzed the damage-induced formation of foci in NCH-H226 cells treated with bleomycin and CBP501 using confocal immunofluorescence microscopy. As shown in FIG. 9a, bleomycin at 0.4 μg/ml only marginally increased foci formation detected by γ-H2AX, NBS1 p-Ser343, ATM p-Ser1981 and SMC1 p-Ser957. Addition of 0.2 or 2 μM CBP501 significantly increased the bleomycin-induced formation of foci. CBP501 alone did not induce foci formation in cells (data not shown).

Enhanced foci formation by CBP501 was further confirmed by quantitative analysis of the foci count in cells. Treated cells were stained with ATM p-Ser1981, DNA-PKcs p-Thr2609, NBS1 p-Ser343, γ-H2AX and SMC1 p-Ser957 9 hr or 12 hr after is the treatment in the kinetic analysis, and the number of foci counted. Foci numbers significantly increased in cells treated with bleomycin plus CBP501 compared to bleomycin alone (FIG. 9b). The kinetics of the foci formation as determined by SMC p-Ser957 was slower than that determined by other factors.

### Experimental Example 4

### CBP501 Enhances Checkpoint Signals in Bleomycin and Cisplatin Treated Cells

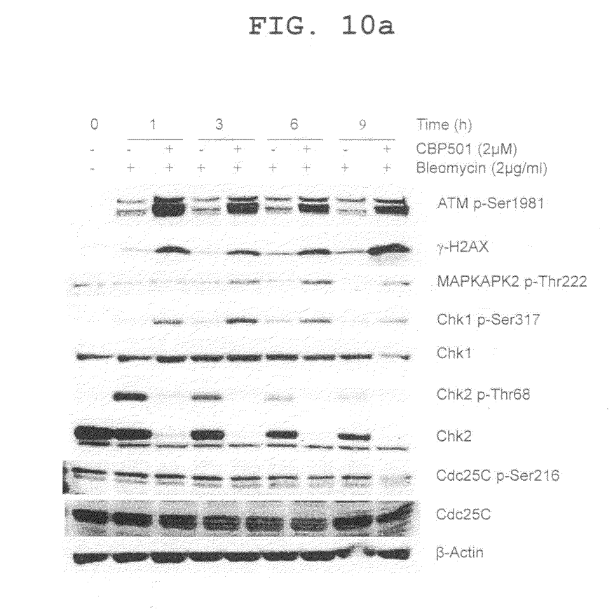

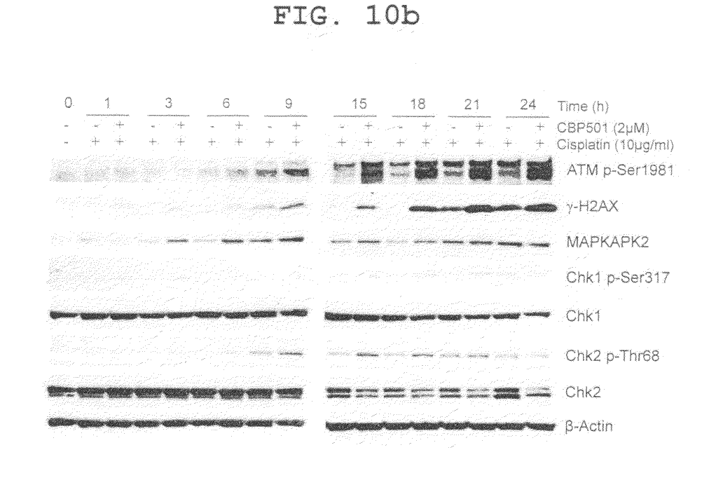

The above experiments indicate that the DNA damage caused by cisplatin and bleomycin was increased by treatment with CBP501. The present inventor next assessed the status of the checkpoint signal proteins by western blot (FIG. 10a). Treatment of NCI-H226 cells with bleomycin (2 μg/ml) induced phosphorylation of the upstream checkpoint signal proteins, such as ATM (Ser1981) and H2AX, as well as downstream checkpoint proteins CHK1 (Ser317) and CHK2 (Thr68). Co-treatment with 2 μm of CBP501 dramatically increased phosphorylation of ATM (Ser1981), H2AX, and CHK1 (Ser317) as well as enhanced the phosphorylation of MAPKAPK2 (Thr222), indicating that the checkpoint signals were enhanced by the addition of CBP501. Strikingly, we observed a dramatic disappearance of CHK2 upon co-treatment of CBP501 with bleomycin. Other experiments showed a similar abolishment of CHK2 with significantly high levels of bleomycin (data not shown). At 9 hr of treatment with bleomycin and CBP501, CHK1 protein levels began to decrease, compared to CHK1 levels with bleomycin treatment alone. Phosphorylation of CDC25C (Ser 216) began to decrease 6 hr after treatment with both bleomycin and CBP501, and continued decrease until 9 hr after treatment, consistent with results from a previous study with Jurkat cells (non-patent literature 14).

We performed a similar analysis using cisplatin treatment (FIG. 10b). Co-treatment of CBP501 with cisplatin increased the phosphorylation of ATM (Ser1981), H2AX, MAPKAPK2 (Thr222), CHK2 (Thr68), and CHK1 (Ser317), though at much weaker levels and showing a slower time course than bleomycin and CBP501. Since at least ATM p-Ser1981 and γ-H2A are indicative of double strand breaks, this suggests secondary double strand breaks. CBP501 itself did not change the phosphorylation status or the protein levels of the checkpoint proteins tested above (data not shown).

### Experimental Example 5

### CBP501 Enhances the Cytotoxicity of Cisplatin in Mesothelioma Cell Lines

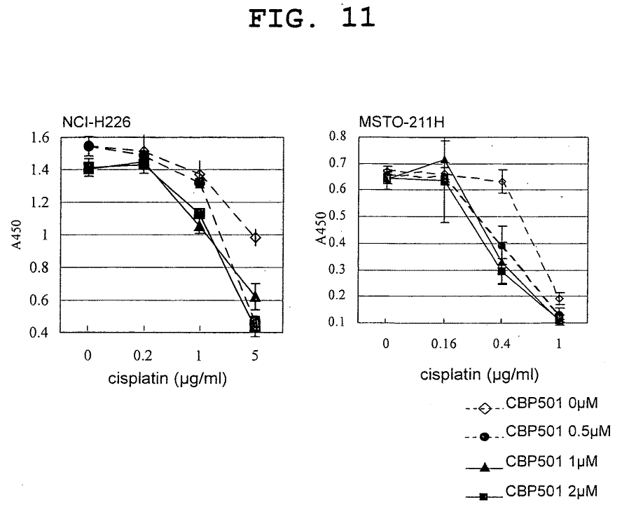

As described above, CBP501 enhanced the amount of DNA damage and damage response of cells treated with bleomycin or cisplatin. To confirm that the cytotoxicity of cisplatin was increased by the addition of CBP501, WST analysis was performed on NCI-H226 and MSTO-211H cells. As shown in FIG. 11, the cytotoxicity of cisplatin was increased by the addition of CBP501 in both cell lines.

### Experimental Example 6

### CBP501 Enhanced Anti-Tumor Activity of Cisplatin In Vivo

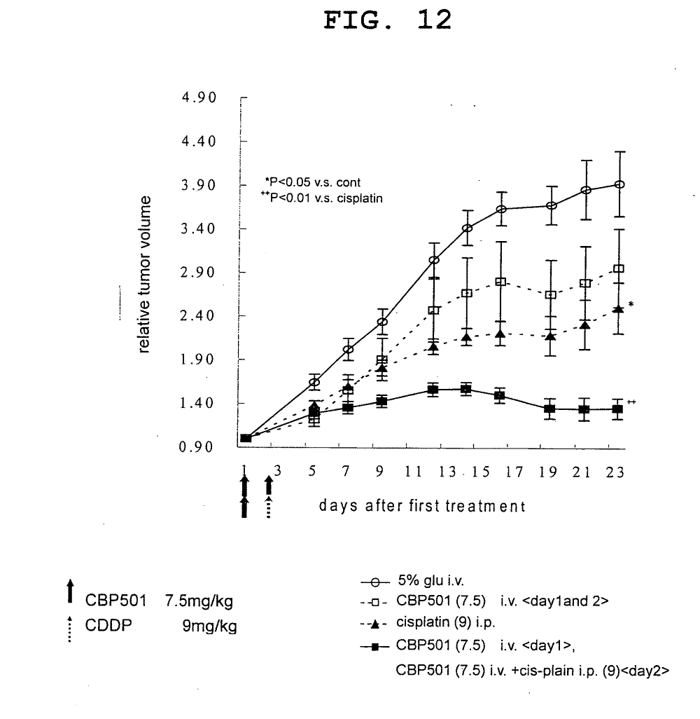

The anti-tumor activity of cisplatin, CBP501 and the combination was examined by employing the subcutaneous tumor xenograft model in SCID mice. As shown in FIG. 12, intra-venous bolus injection of 7.5 mg/kg of CBP501 or 9 mg/kg of cisplatin suppressed tumor growth, and the combination of the two further suppressed tumor growth. CBP501 tissue distribution was analyzed using ³H-labelled CBP501 in the same xenograft model, as well as in the MSTO-211H xenograft model. CBP501 was distributed among various organs including xenograft tumors of NCI-H226 and MSTO-211H (Table 15). The dose levels, approximately 1 to 2 μM, were consistent with the levels of CBP501 that showed in vitro activities.

Concentration of CBP501 in various tissues and the subcutaneous tumor xenograft. Levels were detected using ³H-labelled CBP501 4 hr after the bolus injection of CBP501 once at 7.5 mg/kg.

TABLE 15

Radioactivity concentration
(ng eq. of CBP501/g or mL +/− SD)

| Tissue | NCI-H226 | MSTO-211H |
|---|---|---|
| Plasma | 81.64 ± 8.34 | 61.20 ± 10.54 |
| Heart | 1736.39 ± 122.49 | 1454.63 ± 44.27 |
| Lung | 2323.22 ± 168.98 | 1882.29 ± 168.88 |
| Liver | 39160.82 ± 646.25 | 40010.39 ± 991.96 |
| Kidney | 4804.17 ± 332.21 | 4009.81 ± 22.39 |
| Spleen | 6097.37 ± 80.17 | 4975.78 ± 268.83 |
| Tumor | 2165.19 ± 595.10 | 1079.04 ± 144.19 |

Data are expressed as the mean values ± SD of data from three animals.

### CONCLUSION

CBP501 has a G2 checkpoint abrogating activity. This effect was associated with reduced phosphorylation at serine 216 of CDC25C at 6 to 9 hr after treatment (non-patent literature 14). Notably, the G2/M phase population increased rather than decreased in response to co-treatment with CBP501 when the dose levels of bleomycin or cisplatin were too low to induce cell accumulation in G2/M phase. As shown in this report, CBP501 increased DNA damage-induced foci formation (FIG. 9) and the damage signal transduction (FIG. 10a) in bleomycin treated cells, and increased platinum concentration (FIG. 8a), DNA-platinum adduct formation (FIG. 8b) and the damage signal transduction (FIG. 10b) in cisplatin treated cells. Of note, serine 216 phosphorylation of CDC25C was reduced upon co-treatment with BGP501 in bleomycin treated cells (FIG. 10a).

Notably, the present inventor observed a dramatic disappearance of CHK2 upon combined treatment of CBP501 and bleomycin (FIG. 10a). A similar effect was observed in the experiments with cisplatin and CBP501 (FIG. 10b), in which CHK2 protein decreased upon addition of CBP501 after 15 hr of treatment. Reduction of CHK2 in response to DNA damage was first reported by Zhang et al. in 2005 with cisplatin treatment (non-patent literature 16), which was later found to be mediated by phosphorylation of specific sites on CHK2, leading to its ubiquitination and degradation (non-patent literature 17, non-patent literature 18). As the present inventor observed a similar trend using increased doses of bleomycin treatment alone (data not shown), the present inventor speculated that the increased DNA damage in response to CBP501 co-treatment caused the dramatic disappearance of CHK2 in FIG. 10.

Above-mentioned results imply that CBP501 may enhance influx, increase retention or reduce efflux, of bleomycin and cisplatin, however, no common transporter, either influx or efflux, for cisplatin and bleomycin but not for doxorubicine and camptothecine has been found. Moreover, bleomycin causes DNA double strand breaks and cisplatin causes nucleotide adducts or cross-links, which are recognized by different sensor protein complexes and repaired by different repair proteins. However, as indicated in FIG. 10b, cisplatin induced double strand breaks with much slower kinetics than bleomycin treatment, presumably resulting from when damage was repaired or when the replication folk collided with adducts or cross-links in the genome. The augmentation of DNA damage by CBP501 was observed only with bleomycin and cisplatin, and to a lesser extent with carboplatin and oxaliplatin (data not shown), but not with doxorubicin, camptothecin, X-irradiation or 5-FU (data not shown). The mechanism by which CBP501 increases platinum concentration in the cells and enhances bleomycin induced DNA damage is not yet known; however, the activity is prominent and seems relatively specific to cancer cells, as indicated by the in vitro experiments with HUVEC (FIG. 8b), normal human dermal fibroblast and PHA(phytohaemagglutinin)-blast cells (data not shown), and by the low toxicity observed in mice and humans when combined with cisplatin (non-patent literature 15).

CBP501 enhanced the cytotoxicity of cisplatin (FIG. 11) and bleomycin (non-patent literature 14) and suppressed tumor xenograft growth in mice (FIG. 12). Given the Cmax in the human studies (non-patent literature 15), which is approximately 4 μm at the recommended dose, and the concentration of CBP501 in the subcutaneous tumor xenograft model, approximately 1 to 2 μM (Table 15), this activity of CBP501 may be the main mechanism of action in humans. Given that the tissue distribution of CBP501 in the lung was similar to the level in xenograft tumors (Table 15) and all four tested mesothelioma cell lines, NCI-H226 could be a non-small cell lung cancer (NSCLC) derived cells, were sensitive to CBP501, the efficacy of CBP501 in cancers of the lung, such as pleural mesothelioma and NSCLC, should be examined.

### INDUSTRIAL APPLICABILITY

Using CBP501 and DNA damaging agent in combination, more effective anti-cancer treatment causing less side effects can be provided.

1. An acetate salt of a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1).

2. An agent for the prophylaxis or treatment of a cell proliferative disorder, comprising the acetate salt of the peptide compound of claim 1 as an active ingredient.

3-4. (canceled)

5. A method of producing an acetate salt of a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) SEQ ID No:1, comprising performing liquid chromatography using an acetate-containing solvent.

6. An agent for the prophylaxis or treatment of at least one disease selected from endometrial cancer, peritoneal mesothelioma and pericardial mesothelioma, comprising a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof, or a pharmaceutically acceptable salt thereof as an active ingredient.

7. A pharmaceutical composition comprising an acetate salt of a peptide compound comprising sequence: (d-Bpa)(d-Ser) (d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1) and a nucleic acid damaging agent.

8-10. (canceled)

11. The pharmaceutical composition of claim 7, wherein the nucleic acid damaging agent is at least one selected from bleomycins and a platinum-containing drug.

12. The pharmaceutical composition of claim 7, wherein the nucleic acid damaging agent is at least one selected from bleomycin, cisplatin, carboplatin and oxaliplatin.

13. The pharmaceutical composition of claim 7, wherein the nucleic acid damaging agent is cisplatin.

14. The pharmaceutical composition of any one of claims 11 to 13, further comprising pemetrexed.

15. (canceled)

16. The pharmaceutical composition of any one of claims 11 to 13, further comprising gemcitabine.

17. (canceled)

18. An agent for the prophylaxis or treatment of a cell proliferation disorder, comprising a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof as an active ingredient, which is administered simultaneously with or before a nucleic acid damaging agent.

19. The agent of claim 18, wherein the active ingredient is an acetate salt of the peptide.

20-21. (canceled)

22. The agent of claim 18 or 19, wherein the nucleic acid damaging agent is at least one selected from bleomycins and a platinum-containing drug.

23. The agent of claim 18 or 19, wherein the nucleic acid damaging agent is at least one selected from bleomycin, cisplatin, carboplatin and oxaliplatin.

24. The agent of claim 23, wherein the nucleic acid damaging agent is cisplatin.

25. The agent of claim 22, further comprising pemetrexed.

26. The agent of claim 23, further comprising pemetrexed.

27. The agent of claim 22, further comprising gemcitabine.

28. The agent of claim 23, further comprising gemcitabine.

29. An agent for the prophylaxis or treatment of a cell proliferation disorder, comprising a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof as an active ingredient, which is administered after administration of a nucleic acid damaging agent.

30. The agent of claim 29, wherein the active ingredient is an acetate salt of the peptide.

31-32. (canceled)

33. The agent of claim 29 or 30, wherein the nucleic acid damaging agent is carboplatin or oxaliplatin.

34. A method for the prophylaxis or treatment of a cell proliferation disorder in a mammal, comprising administering a therapeutically effective amount of a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof to the mammal with or before a nucleic acid damaging agent.

35. The method of claim 34, wherein the peptide compound is administered to the mammal before the nucleic acid damaging agent.

36. The method of claim 34, wherein the active ingredient is an acetate salt of the peptide.

37. The method of any one of claims 34 to 36, wherein the cell proliferative disorder is at least one selected from breast cancer, prostate cancer, pancreas cancer, gastric cancer, lung cancer, pleural mesothelioma, colon cancer, rectal cancer, large bowel cancer, small intestinal cancer, esophageal cancer, duodenal cancer, lingual cancer, pharyngeal cancer, salivary gland cancer, cerebral tumor, schwanoma, liver cancer, kidney cancer, bile duct cancer, endometrial cancer, cervical cancer, uterine body cancer, ovarian cancer, bladder cancer, urethral cancer, skin cancer, angioma, malignant lymphoma, malignant melanoma, thyroid cancer, parathyroid cancer, nasal cancer, paranasal cancer, auditory organ cancer, carcinoma of oral floor, laryngeal cancer, unknown primary cancer, parotid cancer, submandibular cancer, bone tumor, angiofibroma, retinal sarcoma, penile cancer, testicular tumor, pediatric solid cancer, Kaposi's sarcoma, Kaposi's sarcoma resulted from AIDS, tumor of maxillary sinus, fibrous histiocytoma, leiomyosarcoma, rhabdomyosarcoma, multiple myeloma and leukemia.

38. The method of any one of claims 34 to 36, wherein the cell proliferative disorder is at least one selected from endometrial cancer, peritoneal mesothelioma, pericardial mesothelioma, uterine body cancer and ovarian cancer.

39. The method of any one of claims 34 to 36, wherein the nucleic acid damaging agent is at least one selected from bleomycins and a platinum-containing drug.

40. The method of any one of claims 34 to 36, wherein the nucleic acid damaging agent is at least one selected from bleomycin, cisplatin, carboplatin and oxaliplatin.

41. The method of claim 40, wherein the nucleic acid damaging agent is cisplatin.

42. The method of claim 39, further comprising administering pemetrexed.

43. The method of claim 42, wherein the cell proliferative disorder is peritoneal mesothelioma.

44. The method of claim 39, further comprising administering gemcitabine.

45. The method of claim 44, wherein the cell proliferative disorder is pancreas cancer.

46. A method for the prophylaxis or treatment of a cell proliferation disorder in a mammal, comprising performing the following step a) and step b) as one cycle once a week for 3 weeks;
a) administering a therapeutically effective amount of a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof to a mammal by intravenous infusion, and
b) administering a therapeutically effective amount of cisplatin to the mammal after completion of step a).

47. A method for the prophylaxis or treatment of a cell proliferation disorder in a mammal, comprising performing the following step a) and step b) as one cycle once a day for 5 consecutive days;
a) administering a therapeutically effective amount of a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof to a mammal by intravenous infusion, and
b) administering a therapeutically effective amount of cisplatin to the mammal after completion of step a).

48. A method for the prophylaxis or treatment of a cell proliferation disorder in a mammal, comprising performing the following step a)-step c) as one cycle every 3 weeks;
a) administering a therapeutically effective amount of a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof to a mammal by intravenous infusion,
b) administering a therapeutically effective amount of pemetrexed to the mammal after completion of step a), and
c) administering a therapeutically effective amount of cisplatin to the mammal after completion of step b).

49. A method for the prophylaxis or treatment of a cell proliferation disorder in a mammal, comprising administering a therapeutically effective amount of a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof to the mammal after administration of a nucleic acid damaging agent.

50. The method of claim 49, wherein the pharmaceutically acceptable salt is an acetate salt.

51. The method of claim 49, wherein the cell proliferative disorder is at least one selected from breast cancer, prostate cancer, pancreas cancer, gastric cancer, lung cancer, pleural mesothelioma, colon cancer, rectal cancer, large bowel cancer, small intestinal cancer, esophageal cancer, duodenal cancer, lingual cancer, pharyngeal cancer, salivary gland cancer, cerebral tumor, schwanoma, liver cancer, kidney cancer, bile duct cancer, endometrial cancer, cervical cancer, uterine body cancer, ovarian cancer, bladder cancer, urethral cancer, skin cancer, angioma, malignant lymphoma, malignant melanoma, thyroid cancer, parathyroid cancer, nasal cancer, paranasal cancer, auditory organ cancer, carcinoma of oral floor, laryngeal cancer, unknown primary cancer, parotid cancer, submandibular cancer, bone tumor, angiofibroma, retinal sarcoma, penile cancer, testicular tumor, pediatric solid cancer, Kaposi's sarcoma, Kaposi's sarcoma resulted from AIDS, tumor of maxillary sinus, fibrous histiocytoma, leiomyosarcoma, rhabdomyosarcoma, multiple myeloma and leukemia.

52. The method of claim 49, wherein the cell proliferative disorder is at least one selected from endometrial cancer, peritoneal mesothelioma, pericardial mesothelioma, uterine body cancer and ovarian cancer.

53. The method of claim 49, wherein the nucleic acid damaging agent is at least one selected from carboplatin and oxaliplatin.

54. An agent for potentiating a cell proliferation suppressive action of a platinum-containing preparation, comprising a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof as an active ingredient.

55. The agent of claim 54, wherein the platinum-containing preparation is cisplatin, carboplatin or oxaliplatin.

56. The agent of claim 54, wherein the platinum-containing preparation is cisplatin.

57. The agent of any one of claims 54 to 56, further comprising pemetrexed.

58. The agent of any one of claims 54 to 56, further comprising gemcitabine.

59. A method for potentiating a cell proliferation suppressive action of a platinum-containing preparation, comprising administering, to a mammal, a therapeutically effective amount of a peptide compound comprising sequence: (d-Bpa)(d-Ser)(d-Trp)(d-Ser)(d-Phe-2,3,4,5,6-F)(d-Cha)(d-Arg)(d-Arg)(d-Arg)(d-Gln)(d-Arg)(d-Arg) (SEQ ID NO:1), a prodrug thereof or a pharmaceutically acceptable salt thereof as an active ingredient.

60. The method of claim 59, wherein the platinum-containing preparation is cisplatin, carboplatin or oxaliplatin.

61. The method of claim 59, wherein the platinum-containing preparation is cisplatin.

62. The method of any one of claims 59 to 61, further comprising administering pemetrexed.

63. The method of any one of claims 59 to 61, further comprising administering gemcitabine.
